(19) United States
(12) Patent Application Publication
(10) Pub. No.: US 2026/0226541 A1
(43) Pub. Date: Aug. 6, 2026

(54) USE OF SNP SITE COMBINATION OF LITOPENAEUS VANNAMEI IN TRAIT EVALUATION OF MIXED-FAMILY CULTURE, AND PROBE AND KIT

(71) Applicant: YELLOW SEA FISHERIES RESEARCH INSTITUTE (YSFRI), CHINESE ACADEMY OF FISHERY SCIENCES (CAFS), Qingdao (CN)

(72) Inventors: Sheng LUAN, Qingdao (CN); Mianyu LIU, Qingdao (CN); Jie KONG, Qingdao (CN); Qiang FU, Qingdao (CN); Ping DAI, Qingdao (CN); Xianhong MENG, Qingdao (CN); Kun LUO, Qingdao (CN); Juan SUI, Qingdao (CN); Jian TAN, Qingdao (CN); Baolong CHEN, Qingdao (CN); Xupeng LI, Qingdao (CN); Guangfeng QIANG, Qingdao (CN)

(21) Appl. No.: 19/634,491

(22) Filed: Mar. 31, 2026

Related U.S. Application Data
(63) Continuation of application No. PCT/CN2024/109076, filed on Aug. 1, 2024.

(30) Foreign Application Priority Data
Apr. 11, 2024 (CN) 202410431050.9

(51) Int. Cl. C12Q 1/6869; C12Q 1/6806; C12Q 1/6809; C12Q 1/6888

(52) U.S. Cl. CPC C12Q 1/6869; C12Q 1/6806; C12Q 1/6809; C12Q 1/6888; C12Q 2600/124; C12Q 2600/156

(57) ABSTRACT

The use of an SNP site combination of Litopenaeus vannamei in trait evaluation of mixed-family culture, a probe and a kit. The SNP site combination of Litopenaeus vannamei comprises 1125 SNP sites, wherein the physical positions are determined on the basis of alignment with a reference genome sequence of Litopenaeus vannamei. Also disclosed are a molecular probe for capturing the SNP site combination and a kit comprising the molecular probe. By means of using the SNP site combination for paternity identification, the present invention can achieve the mixed culture of individuals of different families in the early stage, so that the common environmental effect caused by separate family culture in the early stage is effectively reduced, and the accuracy of a trait evaluation result of mixed culture is improved; moreover, the genetic relationship between individuals can be accurately calculated. Compared with physical marks, the present invention avoids the phenomena of falling and dislocation of marks on individuals and human identification errors, thus obtaining accurate pedigrees.

### TECHNICAL FIELD

The present invention relates to the technical field of Litopenaeus vannamei breeding, particularly to an application of an SNP site combination in family mixed breeding trait evaluation of Litopenaeus vannamei, and a probe and kit therefor.

### BACKGROUND

Litopenaeus vannamei is the shrimp species with the largest global aquaculture production, characterized by rapid growth, notable disease resistance, and significant economic benefits from breeding. Litopenaeus vannamei was introduced to China in 1988 and, following the resolution of large-scale breeding constraints around 2000, was rapidly adopted nationwide. Over more than three decades of development, culture methods for Litopenaeus vannamei have matured and production has steadily increased year by year, exceeding 6.3 million tons globally in 2021. High-quality broodstock underpins improvements in industry performance, and large-scale family breeding methods are a principle technical approach for Litopenaeus vannamei broodstock selection.

In traditional large-scale family breeding programs, mixed-family breeding is used to evaluate traits such as growth and survival across multiple families. Family identity is typically maintained by physical tagging, for example by applying distinct color combinations of Visible Implant Elastomer (VIE) to identify individual family members and track lineage. To permit reliable physical tagging, family groups must be separately cultured for 50-70 days until juveniles grow to a suitable size. Extended separate culture increases environmental heterogeneity among families, introducing common environmental effects that can bias mixed-family trait evaluations. Physical tags are also subject to detachment, misapplication, and human reading errors, which can lead to pedigree misassignment and further reduce evaluation accuracy.

### SUMMARY

The objective of the present invention is to provide an application of an SNP site combination in family mixed breeding trait evaluation of Litopenaeus vannamei, and a probe and kit therefor, aimed at resolving technical problems associated with physical marker-based large-scale family breeding techniques that compromise the accuracy of Litopenaeus vannamei family mixed breeding trait evaluation. Such problems result from common environmental effects introduced by separate rearing of family groups and from individual marker detachment, misplacement, and human identification errors.

In one aspect, the present invention provides an application of an SNP site combination of Litopenaeus vannamei in family mixed breeding trait evaluation, wherein the SNP site combination comprises 1,125 SNP sites. The physical positions of the SNP sites are determined by comparison to the Litopenaeus vannamei reference genome sequence. Information for the SNP sites is shown in Table 1, wherein “ID” is the site identifier, “Scaffold” is the name of the scaffold in the Litopenaeus vannamei reference genome on which the site is located, “Position” is the physical coordinate of the site on the scaffold, and “Ref” is the reference genome base at the site for Litopenaeus vannamei.

TABLE 1
Information for the SNP Sites

| ID | Scaffold | Position | Ref |
|---|---|---|---|
| A1 | NW_020868289.1 | 207791 | G |
| A2 | NW_020868292.1 | 51443 | C |
| A3 | NW_020868293.1 | 255982 | G |
| A4 | NW_020868301.1 | 214864 | T |
| A5 | NW_020868304.1 | 155318 | G |
| A6 | NW_020868305.1 | 720780 | C |
| A7 | NW_020868306.1 | 207725 | A |
| A8 | NW_020868307.1 | 582033 | G |
| A9 | NW_020868311.1 | 501920 | C |
| A10 | NW_020868316.1 | 42724 | T |
| A11 | NW_020868318.1 | 292168 | A |
| A12 | NW_020868324.1 | 94414 | C |
| A13 | NW_020868328.1 | 310383 | T |
| A14 | NW_020868334.1 | 184665 | A |
| A15 | NW_020868335.1 | 210210 | C |
| A16 | NW_020868337.1 | 214071 | G |
| A17 | NW_020868341.1 | 493235 | A |
| A18 | NW_020868344.1 | 222217 | T |
| A19 | NW_020868345.1 | 326144 | C |
| A20 | NW_020868351.1 | 136314 | T |
| A21 | NW_020868353.1 | 384502 | T |
| A22 | NW_020868357.1 | 682725 | T |
| A23 | NW_020868360.1 | 335685 | C |
| A24 | NW_020868364.1 | 251991 | G |
| A25 | NW_020868366.1 | 81392 | C |
| A26 | NW_020868369.1 | 13259 | G |
| A27 | NW_020868377.1 | 169009 | A |
| A28 | NW_020868379.1 | 204340 | C |
| A29 | NW_020868386.1 | 33176 | A |
| A30 | NW_020868390.1 | 130449 | A |
| A31 | NW_020868393.1 | 310814 | C |
| A32 | NW_020868394.1 | 328073 | T |
| A33 | NW_020868397.1 | 96282 | G |
| A34 | NW_020868406.1 | 3844 | G |
| A35 | NW_020868415.1 | 1652844 | C |
| A36 | NW_020868416.1 | 212490 | G |
| A37 | NW_020868429.1 | 333337 | C |
| A38 | NW_020868431.1 | 98599 | C |
| A39 | NW_020868436.1 | 216158 | C |
| A40 | NW_020868439.1 | 337192 | T |
| A41 | NW_020868441.1 | 655324 | T |
| A42 | NW_020868444.1 | 944654 | T |
| A43 | NW_020868450.1 | 175085 | G |
| A44 | NW_020868453.1 | 712720 | T |
| A45 | NW_020868455.1 | 160936 | C |
| A46 | NW_020868456.1 | 12986 | G |
| A47 | NW_020868458.1 | 795878 | C |
| A48 | NW_020868461.1 | 49289 | C |
| A49 | NW_020868467.1 | 243436 | G |
| A50 | NW_020868468.1 | 994083 | A |
| A51 | NW_020868479.1 | 38677 | T |
| A52 | NW_020868481.1 | 510733 | C |
| A53 | NW_020868484.1 | 19731 | A |
| A54 | NW_020868486.1 | 181933 | C |
| A55 | NW_020868495.1 | 169988 | T |
| A56 | NW_020868506.1 | 263825 | T |
| A57 | NW_020868506.1 | 1054292 | T |
| A58 | NW_020868512.1 | 955288 | A |
| A59 | NW_020868523.1 | 464246 | A |
| A60 | NW_020868534.1 | 22047 | T |
| A61 | NW_020868537.1 | 421538 | A |
| A62 | NW_020868538.1 | 321994 | C |
| A63 | NW_020868539.1 | 201636 | G |
| A64 | NW_020868542.1 | 427985 | G |
| A65 | NW_020868547.1 | 275291 | G |
| A66 | NW_020868548.1 | 338068 | T |
| A67 | NW_020868553.1 | 202039 | A |
| A68 | NW_020868557.1 | 44933 | G |
| A69 | NW_020868557.1 | 884071 | A |
| A70 | NW_020868561.1 | 60426 | T |
| A71 | NW_020868562.1 | 39367 | A |
| A72 | NW_020868566.1 | 70832 | G |
| A73 | NW_020868568.1 | 620385 | C |
| A74 | NW_020868571.1 | 327147 | C |
| A75 | NW_020868578.1 | 487353 | A |
| A76 | NW_020868579.1 | 99291 | A |
| A77 | NW_020868581.1 | 1175016 | T |
| A78 | NW_020868582.1 | 77505 | T |
| A79 | NW_020868588.1 | 53104 | G |
| A80 | NW_020868590.1 | 166732 | A |
| A81 | NW_020868593.1 | 219106 | T |
| A82 | NW_020868596.1 | 189463 | C |
| A83 | NW_020868596.1 | 400219 | C |
| A84 | NW_020868602.1 | 363304 | C |
| A85 | NW_020868605.1 | 384094 | C |
| A86 | NW_020868607.1 | 690423 | A |
| A87 | NW_020868608.1 | 315923 | A |
| A88 | NW_020868610.1 | 124096 | G |
| A89 | NW_020868611.1 | 658095 | C |
| A90 | NW_020868617.1 | 126431 | A |
| A91 | NW_020868624.1 | 544119 | T |
| A92 | NW_020868626.1 | 18002 | C |
| A93 | NW_020868628.1 | 455458 | C |
| A94 | NW_020868629.1 | 448174 | A |
| A95 | NW_020868631.1 | 381221 | G |
| A96 | NW_020868637.1 | 10141 | T |
| A97 | NW_020868637.1 | 826121 | C |
| A98 | NW_020868641.1 | 543888 | C |
| A99 | NW_020868646.1 | 984476 | A |
| A100 | NW_020868653.1 | 385080 | T |
| A101 | NW_020868658.1 | 157072 | C |
| A102 | NW_020868662.1 | 201191 | G |
| A103 | NW_020868666.1 | 3747 | C |
| A104 | NW_020868667.1 | 314864 | A |
| A105 | NW_020868668.1 | 170556 | T |
| A106 | NW_020868669.1 | 444922 | T |
| A107 | NW_020868671.1 | 56108 | C |
| A108 | NW_020868672.1 | 1050794 | G |
| A109 | NW_020868675.1 | 714980 | A |
| A110 | NW_020868678.1 | 474798 | T |
| A111 | NW_020868680.1 | 240054 | A |
| A112 | NW_020868681.1 | 93241 | C |
| A113 | NW_020868685.1 | 56549 | A |
| A114 | NW_020868686.1 | 375097 | T |
| A115 | NW_020868688.1 | 544675 | T |
| A116 | NW_020868695.1 | 15856 | A |
| A117 | NW_020868700.1 | 560346 | T |
| A118 | NW_020868703.1 | 28437 | T |
| A119 | NW_020868707.1 | 513384 | T |
| A120 | NW_020868714.1 | 369543 | C |
| A121 | NW_020868718.1 | 424008 | A |
| A122 | NW_020868733.1 | 733573 | C |
| A123 | NW_020868737.1 | 1236920 | T |
| A124 | NW_020868740.1 | 204092 | A |
| A125 | NW_020868745.1 | 274449 | G |
| A126 | NW_020868748.1 | 418551 | C |
| A127 | NW_020868752.1 | 697563 | A |
| A128 | NW_020868753.1 | 255840 | G |
| A129 | NW_020868760.1 | 67162 | C |
| A130 | NW_020868770.1 | 87546 | C |
| A131 | NW_020868772.1 | 349040 | A |
| A132 | NW_020868782.1 | 1104469 | A |
| A133 | NW_020868784.1 | 41074 | G |
| A134 | NW_020868786.1 | 235309 | T |
| A135 | NW_020868790.1 | 963672 | T |
| A136 | NW_020868792.1 | 706674 | G |
| A137 | NW_020868795.1 | 18926 | T |
| A138 | NW_020868801.1 | 525112 | C |
| A139 | NW_020868804.1 | 600802 | A |
| A140 | NW_020868808.1 | 45427 | C |
| A141 | NW_020868810.1 | 900504 | T |
| A142 | NW_020868812.1 | 100914 | A |
| A143 | NW_020868818.1 | 356954 | G |
| A144 | NW_020868823.1 | 284130 | A |
| A145 | NW_020868825.1 | 829040 | T |
| A146 | NW_020868830.1 | 797200 | T |
| A147 | NW_020868835.1 | 410664 | G |
| A148 | NW_020868837.1 | 447441 | C |
| A149 | NW_020868847.1 | 13463 | A |
| A150 | NW_020868854.1 | 183182 | A |
| A151 | NW_020868857.1 | 566613 | G |
| A152 | NW_020868865.1 | 87671 | C |
| A153 | NW_020868868.1 | 838369 | T |
| A154 | NW_020868871.1 | 753036 | G |
| A155 | NW_020868874.1 | 499739 | A |
| A156 | NW_020868880.1 | 646919 | C |
| A157 | NW_020868889.1 | 255114 | T |
| A158 | NW_020868893.1 | 161379 | T |
| A159 | NW_020868894.1 | 285175 | C |
| A160 | NW_020868899.1 | 115023 | C |
| A161 | NW_020868905.1 | 584705 | A |
| A162 | NW_020868908.1 | 132232 | T |
| A163 | NW_020868911.1 | 95173 | T |
| A164 | NW_020868912.1 | 131088 | G |
| A165 | NW_020868913.1 | 769402 | A |
| A166 | NW_020868918.1 | 822337 | C |
| A167 | NW_020868919.1 | 487867 | T |
| A168 | NW_020868924.1 | 852288 | C |
| A169 | NW_020868927.1 | 289578 | A |
| A170 | NW_020868928.1 | 114100 | A |
| A171 | NW_020868937.1 | 239783 | C |
| A172 | NW_020868938.1 | 61909 | A |
| A173 | NW_020868944.1 | 23018 | C |
| A174 | NW_020868948.1 | 93510 | G |
| A175 | NW_020868949.1 | 769795 | T |
| A176 | NW_020868952.1 | 436114 | T |
| A177 | NW_020868954.1 | 14469 | G |
| A178 | NW_020868957.1 | 274325 | G |
| A179 | NW_020868964.1 | 27835 | G |
| A180 | NW_020868965.1 | 244652 | A |
| A181 | NW_020868966.1 | 1244464 | G |
| A182 | NW_020868967.1 | 21206 | T |
| A183 | NW_020868968.1 | 213316 | A |
| A184 | NW_020868971.1 | 212365 | T |
| A185 | NW_020868972.1 | 444904 | T |
| A186 | NW_020868973.1 | 445882 | C |
| A187 | NW_020868980.1 | 137253 | A |
| A188 | NW_020868983.1 | 355322 | C |
| A189 | NW_020868989.1 | 242600 | T |
| A190 | NW_020868990.1 | 212634 | T |
| A191 | NW_020868995.1 | 663455 | T |
| A192 | NW_020869007.1 | 190560 | T |
| A193 | NW_020869008.1 | 79923 | T |
| A194 | NW_020869010.1 | 515813 | T |
| A195 | NW_020869012.1 | 238833 | G |
| A196 | NW_020869018.1 | 5436 | G |
| A197 | NW_020869026.1 | 236454 | C |
| A198 | NW_020869029.1 | 842453 | T |
| A199 | NW_020869030.1 | 77495 | A |
| A200 | NW_020869032.1 | 111693 | C |
| A201 | NW_020869035.1 | 186364 | G |
| A202 | NW_020869038.1 | 668187 | T |
| A203 | NW_020869040.1 | 90679 | T |
| A204 | NW_020869050.1 | 1027398 | G |
| A205 | NW_020869050.1 | 2515901 | A |
| A206 | NW_020869052.1 | 135902 | A |
| A207 | NW_020869053.1 | 95158 | T |
| A208 | NW_020869057.1 | 566442 | C |
| A209 | NW_020869059.1 | 150208 | A |
| A210 | NW_020869060.1 | 385541 | T |
| A211 | NW_020869061.1 | 16172 | A |
| A212 | NW_020869061.1 | 984347 | A |
| A213 | NW_020869062.1 | 12095 | T |
| A214 | NW_020869063.1 | 15746 | G |
| A215 | NW_020869065.1 | 151494 | T |
| A216 | NW_020869072.1 | 630097 | A |
| A217 | NW_020869079.1 | 85340 | G |
| A218 | NW_020869081.1 | 96947 | G |
| A219 | NW_020869083.1 | 285832 | G |
| A220 | NW_020869090.1 | 383714 | G |
| A221 | NW_020869091.1 | 120317 | A |
| A222 | NW_020869093.1 | 256906 | C |
| A223 | NW_020869099.1 | 356862 | T |
| A224 | NW_020869103.1 | 304717 | T |
| A225 | NW_020869108.1 | 503392 | T |
| A226 | NW_020869111.1 | 495551 | T |
| A227 | NW_020869117.1 | 68334 | T |
| A228 | NW_020869118.1 | 587902 | T |
| A229 | NW_020869118.1 | 1339586 | G |
| A230 | NW_020869122.1 | 295995 | A |
| A231 | NW_020869124.1 | 969660 | A |
| A232 | NW_020869126.1 | 626743 | A |
| A233 | NW_020869127.1 | 288407 | T |
| A234 | NW_020869128.1 | 920132 | G |
| A235 | NW_020869130.1 | 477325 | A |
| A236 | NW_020869133.1 | 331647 | T |
| A237 | NW_020869133.1 | 1968767 | T |
| A238 | NW_020869146.1 | 68586 | T |
| A239 | NW_020869147.1 | 1519380 | C |
| A240 | NW_020869148.1 | 1045739 | T |
| A241 | NW_020869150.1 | 197717 | T |
| A242 | NW_020869152.1 | 468833 | T |
| A243 | NW_020869154.1 | 315791 | A |
| A244 | NW_020869157.1 | 258113 | T |
| A245 | NW_020869159.1 | 74009 | A |
| A246 | NW_020869160.1 | 178528 | A |
| A247 | NW_020869163.1 | 267396 | A |
| A248 | NW_020869165.1 | 45823 | C |
| A249 | NW_020869172.1 | 401640 | A |
| A250 | NW_020869174.1 | 496874 | C |
| A251 | NW_020869178.1 | 291562 | G |
| A252 | NW_020869180.1 | 102132 | A |
| A253 | NW_020869182.1 | 57276 | A |
| A254 | NW_020869184.1 | 131898 | G |
| A255 | NW_020869186.1 | 279482 | A |
| A256 | NW_020869190.1 | 1248409 | T |
| A257 | NW_020869194.1 | 579276 | A |
| A258 | NW_020869195.1 | 538066 | T |
| A259 | NW_020869197.1 | 278226 | C |
| A260 | NW_020869202.1 | 167338 | T |
| A261 | NW_020869204.1 | 88350 | C |
| A262 | NW_020869206.1 | 467618 | C |
| A263 | NW_020869209.1 | 88840 | T |
| A264 | NW_020869211.1 | 504632 | T |
| A265 | NW_020869213.1 | 598646 | C |
| A266 | NW_020869215.1 | 191818 | G |
| A267 | NW_020869216.1 | 325218 | T |
| A268 | NW_020869222.1 | 203432 | A |
| A269 | NW_020869224.1 | 44463 | C |
| A270 | NW_020869225.1 | 194229 | T |
| A271 | NW_020869230.1 | 319801 | A |
| A272 | NW_020869235.1 | 619438 | A |
| A273 | NW_020869245.1 | 29844 | C |
| A274 | NW_020869247.1 | 178195 | G |
| A275 | NW_020869250.1 | 533141 | C |
| A276 | NW_020869252.1 | 346320 | A |
| A277 | NW_020869259.1 | 279373 | T |
| A278 | NW_020869262.1 | 682757 | A |
| A279 | NW_020869262.1 | 2095888 | A |
| A280 | NW_020869263.1 | 287465 | G |
| A281 | NW_020869265.1 | 18189 | C |
| A282 | NW_020869267.1 | 367142 | T |
| A283 | NW_020869273.1 | 234389 | T |
| A284 | NW_020869276.1 | 220360 | A |
| A285 | NW_020869278.1 | 295920 | T |
| A286 | NW_020869282.1 | 185275 | G |
| A287 | NW_020869283.1 | 440986 | A |
| A288 | NW_020869284.1 | 490196 | T |
| A289 | NW_020869285.1 | 157686 | G |
| A290 | NW_020869288.1 | 429412 | T |
| A291 | NW_020869289.1 | 274845 | G |
| A292 | NW_020869295.1 | 605291 | C |
| A293 | NW_020869298.1 | 143780 | T |
| A294 | NW_020869300.1 | 232544 | T |
| A295 | NW_020869304.1 | 140044 | G |
| A296 | NW_020869305.1 | 240558 | A |
| A297 | NW_020869308.1 | 4607 | T |
| A298 | NW_020869308.1 | 1506719 | T |
| A299 | NW_020869310.1 | 357756 | G |
| A300 | NW_020869311.1 | 73517 | T |
| A301 | NW_020869313.1 | 456152 | C |
| A302 | NW_020869318.1 | 483958 | A |
| A303 | NW_020869321.1 | 403266 | T |
| A304 | NW_020869323.1 | 337949 | C |
| A305 | NW_020869324.1 | 10197 | G |
| A306 | NW_020869325.1 | 283412 | A |
| A307 | NW_020869327.1 | 132353 | C |
| A308 | NW_020869329.1 | 61020 | C |
| A309 | NW_020869332.1 | 545888 | C |
| A310 | NW_020869342.1 | 381408 | T |
| A311 | NW_020869346.1 | 33513 | T |
| A312 | NW_020869353.1 | 323536 | A |
| A313 | NW_020869354.1 | 38988 | T |
| A314 | NW_020869355.1 | 479221 | T |
| A315 | NW_020869356.1 | 46448 | A |
| A316 | NW_020869371.1 | 682895 | A |
| A317 | NW_020869373.1 | 130688 | A |
| A318 | NW_020869383.1 | 180097 | A |
| A319 | NW_020869389.1 | 390875 | A |
| A320 | NW_020869391.1 | 761833 | C |
| A321 | NW_020869393.1 | 19476 | G |
| A322 | NW_020869396.1 | 13983 | T |
| A323 | NW_020869401.1 | 414457 | C |
| A324 | NW_020869405.1 | 147861 | A |
| A325 | NW_020869424.1 | 289967 | T |
| A326 | NW_020869427.1 | 305167 | T |
| A327 | NW_020869429.1 | 1313885 | T |
| A328 | NW_020869431.1 | 314917 | A |
| A329 | NW_020869433.1 | 1151492 | A |
| A330 | NW_020869440.1 | 573441 | G |
| A331 | NW_020869449.1 | 129956 | G |
| A332 | NW_020869451.1 | 107123 | C |
| A333 | NW_020869455.1 | 153372 | T |
| A334 | NW_020869458.1 | 1530425 | G |
| A335 | NW_020869459.1 | 241467 | C |
| A336 | NW_020869464.1 | 196068 | G |
| A337 | NW_020869476.1 | 67798 | A |
| A338 | NW_020869481.1 | 435324 | G |
| A339 | NW_020869482.1 | 450092 | G |
| A340 | NW_020869492.1 | 232251 | A |
| A341 | NW_020869494.1 | 187744 | T |
| A342 | NW_020869495.1 | 609983 | A |
| A343 | NW_020869497.1 | 754535 | A |
| A344 | NW_020869498.1 | 108520 | A |
| A345 | NW_020869500.1 | 547421 | G |
| A346 | NW_020869513.1 | 68173 | A |
| A347 | NW_020869515.1 | 114840 | C |
| A348 | NW_020869516.1 | 252984 | T |
| A349 | NW_020869520.1 | 109706 | T |
| A350 | NW_020869521.1 | 90930 | T |
| A351 | NW_020869523.1 | 128332 | A |
| A352 | NW_020869524.1 | 112844 | G |
| A353 | NW_020869525.1 | 105077 | A |
| A354 | NW_020869527.1 | 1622985 | G |
| A355 | NW_020869529.1 | 673491 | T |
| A356 | NW_020869531.1 | 398475 | C |
| A357 | NW_020869534.1 | 76100 | T |
| A358 | NW_020869537.1 | 156702 | T |
| A359 | NW_020869539.1 | 754062 | A |
| A360 | NW_020869540.1 | 457209 | A |
| A361 | NW_020869543.1 | 89233 | G |
| A362 | NW_020869544.1 | 231706 | A |
| A363 | NW_020869548.1 | 308392 | G |
| A364 | NW_020869550.1 | 104015 | A |
| A365 | NW_020869551.1 | 229330 | T |
| A366 | NW_020869553.1 | 213239 | T |
| A367 | NW_020869557.1 | 465347 | T |
| A368 | NW_020869560.1 | 155981 | T |
| A369 | NW_020869562.1 | 482588 | T |
| A370 | NW_020869563.1 | 63057 | T |
| A371 | NW_020869576.1 | 4657 | T |
| A372 | NW_020869578.1 | 755990 | A |
| A373 | NW_020869588.1 | 69872 | C |
| A374 | NW_020869589.1 | 357316 | G |
| A375 | NW_020869593.1 | 172080 | G |
| A376 | NW_020869594.1 | 79931 | T |
| A377 | NW_020869600.1 | 1131812 | A |
| A378 | NW_020869604.1 | 1160252 | C |
| A379 | NW_020869607.1 | 169135 | A |
| A380 | NW_020869611.1 | 441872 | T |
| A381 | NW_020869612.1 | 249411 | A |
| A382 | NW_020869615.1 | 671966 | T |
| A383 | NW_020869623.1 | 553625 | T |
| A384 | NW_020869625.1 | 643058 | C |
| A385 | NW_020869631.1 | 437006 | G |
| A386 | NW_020869633.1 | 439529 | A |
| A387 | NW_020869635.1 | 285866 | T |
| A388 | NW_020869641.1 | 246268 | A |
| A389 | NW_020869643.1 | 435356 | A |
| A390 | NW_020869644.1 | 261337 | A |
| A391 | NW_020869647.1 | 185967 | A |
| A392 | NW_020869648.1 | 83711 | C |
| A393 | NW_020869652.1 | 1254 | C |
| A394 | NW_020869654.1 | 56215 | T |
| A395 | NW_020869657.1 | 503413 | T |
| A396 | NW_020869660.1 | 995510 | G |
| A397 | NW_020869666.1 | 321912 | T |
| A398 | NW_020869667.1 | 644265 | A |
| A399 | NW_020869668.1 | 133427 | A |
| A400 | NW_020869670.1 | 204712 | T |
| A401 | NW_020869686.1 | 165669 | A |
| A402 | NW_020869688.1 | 625902 | T |
| A403 | NW_020869691.1 | 152555 | C |
| A404 | NW_020869696.1 | 630126 | T |
| A405 | NW_020869697.1 | 58723 | T |
| A406 | NW_020869700.1 | 66246 | T |
| A407 | NW_020869701.1 | 27637 | C |
| A408 | NW_020869702.1 | 29123 | C |
| A409 | NW_020869703.1 | 162375 | C |
| A410 | NW_020869704.1 | 448770 | A |
| A411 | NW_020869708.1 | 40076 | A |
| A412 | NW_020869712.1 | 138510 | T |
| A413 | NW_020869714.1 | 206107 | C |
| A414 | NW_020869716.1 | 92107 | C |
| A415 | NW_020869716.1 | 1012538 | T |
| A416 | NW_020869724.1 | 142019 | A |
| A417 | NW_020869733.1 | 193592 | G |
| A418 | NW_020869735.1 | 510602 | G |
| A419 | NW_020869738.1 | 464112 | T |
| A420 | NW_020869741.1 | 622403 | C |
| A421 | NW_020869744.1 | 308506 | A |
| A422 | NW_020869749.1 | 255697 | C |
| A423 | NW_020869750.1 | 1111542 | G |
| A424 | NW_020869755.1 | 55998 | C |
| A425 | NW_020869760.1 | 602305 | C |
| A426 | NW_020869768.1 | 239388 | C |
| A427 | NW_020869769.1 | 419897 | A |
| A428 | NW_020869770.1 | 196315 | T |
| A429 | NW_020869773.1 | 221972 | A |
| A430 | NW_020869783.1 | 740459 | T |
| A431 | NW_020869785.1 | 455483 | T |
| A432 | NW_020869786.1 | 658075 | T |
| A433 | NW_020869791.1 | 473579 | C |
| A434 | NW_020869797.1 | 395020 | A |
| A435 | NW_020869801.1 | 207821 | A |
| A436 | NW_020869804.1 | 608289 | A |
| A437 | NW_020869806.1 | 649130 | G |
| A438 | NW_020869810.1 | 301451 | G |
| A439 | NW_020869813.1 | 309392 | C |
| A440 | NW_020869817.1 | 537023 | C |
| A441 | NW_020869822.1 | 145802 | C |
| A442 | NW_020869822.1 | 938210 | A |
| A443 | NW_020869823.1 | 227842 | T |
| A444 | NW_020869824.1 | 47037 | G |
| A445 | NW_020869826.1 | 236398 | G |
| A446 | NW_020869829.1 | 120232 | T |
| A447 | NW_020869833.1 | 410023 | T |
| A448 | NW_020869838.1 | 235937 | G |
| A449 | NW_020869845.1 | 158645 | G |
| A450 | NW_020869847.1 | 478253 | A |
| A451 | NW_020869849.1 | 22818 | C |
| A452 | NW_020869849.1 | 275197 | T |
| A453 | NW_020869851.1 | 325471 | C |
| A454 | NW_020869852.1 | 485975 | G |
| A455 | NW_020869857.1 | 93415 | A |
| A456 | NW_020869858.1 | 32972 | A |
| A457 | NW_020869858.1 | 1495939 | C |
| A458 | NW_020869860.1 | 998351 | C |
| A459 | NW_020869861.1 | 108326 | A |
| A460 | NW_020869862.1 | 139247 | T |
| A461 | NW_020869873.1 | 135004 | G |
| A462 | NW_020869878.1 | 86614 | A |
| A463 | NW_020869881.1 | 374778 | T |
| A464 | NW_020869884.1 | 59388 | G |
| A465 | NW_020869885.1 | 271631 | A |
| A466 | NW_020869886.1 | 396151 | G |
| A467 | NW_020869892.1 | 693641 | T |
| A468 | NW_020869894.1 | 949226 | A |
| A469 | NW_020869898.1 | 198025 | T |
| A470 | NW_020869901.1 | 174834 | A |
| A471 | NW_020869903.1 | 605051 | T |
| A472 | NW_020869904.1 | 148553 | A |
| A473 | NW_020869906.1 | 332026 | A |
| A474 | NW_020869908.1 | 369526 | A |
| A475 | NW_020869914.1 | 196197 | T |
| A476 | NW_020869925.1 | 55689 | C |
| A477 | NW_020869926.1 | 193769 | A |
| A478 | NW_020869927.1 | 446694 | T |
| A479 | NW_020869928.1 | 198716 | T |
| A480 | NW_020869929.1 | 368640 | C |
| A481 | NW_020869933.1 | 66316 | T |
| A482 | NW_020869934.1 | 75398 | G |
| A483 | NW_020869935.1 | 210622 | T |
| A484 | NW_020869938.1 | 167689 | T |
| A485 | NW_020869940.1 | 419989 | G |
| A486 | NW_020869942.1 | 137259 | A |
| A487 | NW_020869943.1 | 58979 | A |
| A488 | NW_020869951.1 | 198725 | T |
| A489 | NW_020869955.1 | 518852 | T |
| A490 | NW_020869956.1 | 236663 | T |
| A491 | NW_020869959.1 | 415938 | C |
| A492 | NW_020869964.1 | 186302 | G |
| A493 | NW_020869965.1 | 191840 | T |
| A494 | NW_020869967.1 | 100313 | G |
| A495 | NW_020869970.1 | 186047 | G |
| A496 | NW_020869974.1 | 286513 | A |
| A497 | NW_020869977.1 | 219598 | A |
| A498 | NW_020869982.1 | 756860 | G |
| A499 | NW_020869989.1 | 42865 | A |
| A500 | NW_020869991.1 | 102867 | C |
| A501 | NW_020869992.1 | 236689 | A |
| A502 | NW_020869993.1 | 288192 | T |
| A503 | NW_020869997.1 | 634880 | T |
| A504 | NW_020870005.1 | 329796 | C |
| A505 | NW_020870006.1 | 146826 | A |
| A506 | NW_020870007.1 | 759373 | T |
| A507 | NW_020870013.1 | 95485 | G |
| A508 | NW_020870016.1 | 788722 | G |
| A509 | NW_020870018.1 | 134513 | T |
| A510 | NW_020870019.1 | 564956 | C |
| A511 | NW_020870020.1 | 201467 | G |
| A512 | NW_020870026.1 | 7096 | G |
| A513 | NW_020870026.1 | 1669018 | T |
| A514 | NW_020870026.1 | 2488115 | T |
| A515 | NW_020870028.1 | 978173 | T |
| A516 | NW_020870030.1 | 928403 | T |
| A517 | NW_020870032.1 | 106657 | G |
| A518 | NW_020870033.1 | 112027 | A |
| A519 | NW_020870041.1 | 171362 | G |
| A520 | NW_020870044.1 | 71914 | A |
| A521 | NW_020870048.1 | 142266 | T |
| A522 | NW_020870051.1 | 95835 | T |
| A523 | NW_020870053.1 | 259357 | T |
| A524 | NW_020870058.1 | 179073 | C |
| A525 | NW_020870059.1 | 12160 | A |
| A526 | NW_020870059.1 | 931033 | A |
| A527 | NW_020870061.1 | 548323 | T |
| A528 | NW_020870070.1 | 240228 | T |
| A529 | NW_020870074.1 | 148267 | A |
| A530 | NW_020870075.1 | 549794 | T |
| A531 | NW_020870077.1 | 314531 | G |
| A532 | NW_020870088.1 | 853841 | G |
| A533 | NW_020870091.1 | 1278610 | T |
| A534 | NW_020870092.1 | 412790 | G |
| A535 | NW_020870094.1 | 135391 | C |
| A536 | NW_020870094.1 | 949963 | A |
| A537 | NW_020870095.1 | 421338 | T |
| A538 | NW_020870097.1 | 19715 | T |
| A539 | NW_020870098.1 | 103052 | G |
| A540 | NW_020870100.1 | 904 | T |
| A541 | NW_020870101.1 | 141087 | A |
| A542 | NW_020870106.1 | 1086139 | A |
| A543 | NW_020870107.1 | 77193 | G |
| A544 | NW_020870108.1 | 249667 | A |
| A545 | NW_020870110.1 | 65015 | G |
| A546 | NW_020870116.1 | 490209 | G |
| A547 | NW_020870118.1 | 105026 | A |
| A548 | NW_020870125.1 | 1187034 | T |
| A549 | NW_020870128.1 | 914073 | A |
| A550 | NW_020870129.1 | 381078 | C |
| A551 | NW_020870130.1 | 588755 | G |
| A552 | NW_020870134.1 | 15209 | T |
| A553 | NW_020870136.1 | 28570 | C |
| A554 | NW_020870139.1 | 421228 | A |
| A555 | NW_020870146.1 | 14190 | C |
| A556 | NW_020870146.1 | 640630 | T |
| A557 | NW_020870153.1 | 491192 | A |
| A558 | NW_020870155.1 | 601687 | A |
| A559 | NW_020870157.1 | 41102 | T |
| A560 | NW_020870163.1 | 154714 | G |
| A561 | NW_020870168.1 | 184593 | C |
| A562 | NW_020870169.1 | 210426 | C |
| A563 | NW_020870170.1 | 48911 | G |
| A564 | NW_020870173.1 | 504375 | T |
| A565 | NW_020870179.1 | 15615 | T |
| A566 | NW_020870180.1 | 187409 | A |
| A567 | NW_020870182.1 | 514060 | A |
| A568 | NW_020870183.1 | 448848 | C |
| A569 | NW_020870185.1 | 931364 | C |
| A570 | NW_020870185.1 | 1056348 | G |
| A571 | NW_020870195.1 | 139985 | A |
| A572 | NW_020870202.1 | 609688 | C |
| A573 | NW_020870204.1 | 748743 | T |
| A574 | NW_020870205.1 | 98140 | A |
| A575 | NW_020870207.1 | 281234 | T |
| A576 | NW_020870212.1 | 37595 | T |
| A577 | NW_020870216.1 | 450201 | T |
| A578 | NW_020870217.1 | 52666 | C |
| A579 | NW_020870219.1 | 1200363 | T |
| A580 | NW_020870220.1 | 36279 | G |
| A581 | NW_020870222.1 | 976622 | C |
| A582 | NW_020870226.1 | 261017 | T |
| A583 | NW_020870227.1 | 366896 | G |
| A584 | NW_020870230.1 | 143445 | G |
| A585 | NW_020870233.1 | 140603 | G |
| A586 | NW_020870239.1 | 428948 | A |
| A587 | NW_020870240.1 | 142188 | A |
| A588 | NW_020870241.1 | 902788 | A |
| A589 | NW_020870243.1 | 24603 | T |
| A590 | NW_020870248.1 | 834331 | C |
| A591 | NW_020870271.1 | 532344 | C |
| A592 | NW_020870272.1 | 450477 | G |
| A593 | NW_020870274.1 | 570408 | C |
| A594 | NW_020870276.1 | 441676 | T |
| A595 | NW_020870279.1 | 174080 | A |
| A596 | NW_020870287.1 | 245420 | T |
| A597 | NW_020870292.1 | 32364 | G |
| A598 | NW_020870294.1 | 214578 | T |
| A599 | NW_020870295.1 | 853487 | C |
| A600 | NW_020870296.1 | 442443 | T |
| A601 | NW_020870297.1 | 1570349 | T |
| A602 | NW_020870301.1 | 958295 | C |
| A603 | NW_020870307.1 | 781840 | C |
| A604 | NW_020870308.1 | 314357 | T |
| A605 | NW_020870309.1 | 15767 | T |
| A606 | NW_020870312.1 | 167923 | G |
| A607 | NW_020870314.1 | 124993 | C |
| A608 | NW_020870314.1 | 758904 | G |
| A609 | NW_020870315.1 | 300225 | A |
| A610 | NW_020870315.1 | 1853475 | G |
| A611 | NW_020870319.1 | 82623 | A |
| A612 | NW_020870322.1 | 796636 | G |
| A613 | NW_020870324.1 | 114373 | T |
| A614 | NW_020870325.1 | 236786 | A |
| A615 | NW_020870328.1 | 276258 | T |
| A616 | NW_020870336.1 | 42391 | A |
| A617 | NW_020870338.1 | 86368 | A |
| A618 | NW_020870339.1 | 1083869 | T |
| A619 | NW_020870340.1 | 423934 | T |
| A620 | NW_020870344.1 | 440439 | C |
| A621 | NW_020870345.1 | 53727 | T |
| A622 | NW_020870350.1 | 77221 | T |
| A623 | NW_020870359.1 | 608808 | T |
| A624 | NW_020870362.1 | 211840 | G |
| A625 | NW_020870363.1 | 30185 | T |
| A626 | NW_020870365.1 | 441006 | G |
| A627 | NW_020870368.1 | 1042810 | T |
| A628 | NW_020870373.1 | 263147 | T |
| A629 | NW_020870376.1 | 412874 | T |
| A630 | NW_020870378.1 | 587168 | A |
| A631 | NW_020870379.1 | 511871 | C |
| A632 | NW_020870383.1 | 403961 | G |
| A633 | NW_020870389.1 | 613984 | C |
| A634 | NW_020870392.1 | 175642 | T |
| A635 | NW_020870401.1 | 334819 | T |
| A636 | NW_020870403.1 | 768147 | C |
| A637 | NW_020870406.1 | 84776 | G |
| A638 | NW_020870418.1 | 502089 | T |
| A639 | NW_020870419.1 | 514601 | G |
| A640 | NW_020870426.1 | 848406 | T |
| A641 | NW_020870434.1 | 97027 | T |
| A642 | NW_020870436.1 | 529127 | A |
| A643 | NW_020870437.1 | 661746 | G |
| A644 | NW_020870439.1 | 604056 | G |
| A645 | NW_020870445.1 | 110461 | A |
| A646 | NW_020870449.1 | 788764 | G |
| A647 | NW_020870451.1 | 529328 | A |
| A648 | NW_020870456.1 | 207327 | A |
| A649 | NW_020870462.1 | 71524 | T |
| A650 | NW_020870471.1 | 41622 | T |
| A651 | NW_020870472.1 | 1194008 | G |
| A652 | NW_020870475.1 | 896998 | A |
| A653 | NW_020870482.1 | 203153 | C |
| A654 | NW_020870489.1 | 50576 | G |
| A655 | NW_020870491.1 | 787670 | G |
| A656 | NW_020870492.1 | 628522 | A |
| A657 | NW_020870500.1 | 378380 | G |
| A658 | NW_020870506.1 | 622108 | A |
| A659 | NW_020870509.1 | 158927 | A |
| A660 | NW_020870511.1 | 326844 | A |
| A661 | NW_020870514.1 | 454794 | G |
| A662 | NW_020870523.1 | 109234 | G |
| A663 | NW_020870526.1 | 273784 | G |
| A664 | NW_020870528.1 | 978295 | G |
| A665 | NW_020870529.1 | 1156367 | A |
| A666 | NW_020870531.1 | 1587095 | C |
| A667 | NW_020870536.1 | 173463 | C |
| A668 | NW_020870544.1 | 530509 | T |
| A669 | NW_020870547.1 | 216863 | G |
| A670 | NW_020870553.1 | 189389 | A |
| A671 | NW_020870555.1 | 460066 | G |
| A672 | NW_020870557.1 | 482653 | A |
| A673 | NW_020870561.1 | 8939 | G |
| A674 | NW_020870563.1 | 139314 | G |
| A675 | NW_020870569.1 | 160018 | G |
| A676 | NW_020870572.1 | 412365 | T |
| A677 | NW_020870577.1 | 106097 | T |
| A678 | NW_020870579.1 | 210420 | C |
| A679 | NW_020870588.1 | 190150 | A |
| A680 | NW_020870595.1 | 157158 | A |
| A681 | NW_020870599.1 | 526289 | A |
| A682 | NW_020870603.1 | 249534 | G |
| A683 | NW_020870606.1 | 466247 | T |
| A684 | NW_020870607.1 | 447579 | A |
| A685 | NW_020870609.1 | 153516 | T |
| A686 | NW_020870613.1 | 311909 | T |
| A687 | NW_020870614.1 | 371835 | T |
| A688 | NW_020870615.1 | 171357 | A |
| A689 | NW_020870617.1 | 213457 | T |
| A690 | NW_020870617.1 | 450928 | C |
| A691 | NW_020870621.1 | 17734 | C |
| A692 | NW_020870624.1 | 24937 | T |
| A693 | NW_020870627.1 | 31374 | T |
| A694 | NW_020870630.1 | 812864 | A |
| A695 | NW_020870631.1 | 377778 | A |
| A696 | NW_020870638.1 | 231843 | G |
| A697 | NW_020870643.1 | 140915 | T |
| A698 | NW_020870644.1 | 168572 | G |
| A699 | NW_020870645.1 | 886654 | T |
| A700 | NW_020870646.1 | 300178 | A |
| A701 | NW_020870651.1 | 849718 | A |
| A702 | NW_020870656.1 | 64157 | G |
| A703 | NW_020870657.1 | 476305 | C |
| A704 | NW_020870658.1 | 679268 | G |
| A705 | NW_020870669.1 | 324779 | C |
| A706 | NW_020870673.1 | 522722 | A |
| A707 | NW_020870675.1 | 98201 | A |
| A708 | NW_020870679.1 | 195851 | A |
| A709 | NW_020870681.1 | 210076 | G |
| A710 | NW_020870685.1 | 327314 | A |
| A711 | NW_020870688.1 | 601578 | T |
| A712 | NW_020870689.1 | 792452 | T |
| A713 | NW_020870699.1 | 1258808 | T |
| A714 | NW_020870703.1 | 261441 | A |
| A715 | NW_020870704.1 | 208885 | C |
| A716 | NW_020870707.1 | 623747 | G |
| A717 | NW_020870710.1 | 24937 | A |
| A718 | NW_020870711.1 | 779232 | G |
| A719 | NW_020870724.1 | 36898 | A |
| A720 | NW_020870725.1 | 474640 | T |
| A721 | NW_020870734.1 | 1438006 | T |
| A722 | NW_020870736.1 | 282833 | A |
| A723 | NW_020870737.1 | 340464 | G |
| A724 | NW_020870746.1 | 127976 | C |
| A725 | NW_020870747.1 | 644055 | T |
| A726 | NW_020870750.1 | 123378 | A |
| A727 | NW_020870754.1 | 43620 | G |
| A728 | NW_020870755.1 | 48364 | T |
| A729 | NW_020870758.1 | 134255 | A |
| A730 | NW_020870760.1 | 98156 | C |
| A731 | NW_020870761.1 | 80236 | T |
| A732 | NW_020870761.1 | 731915 | A |
| A733 | NW_020870762.1 | 42894 | C |
| A734 | NW_020870763.1 | 196953 | T |
| A735 | NW_020870764.1 | 371038 | A |
| A736 | NW_020870771.1 | 263880 | G |
| A737 | NW_020870777.1 | 904225 | C |
| A738 | NW_020870779.1 | 121363 | A |
| A739 | NW_020870781.1 | 1006246 | A |
| A740 | NW_020870786.1 | 549851 | C |
| A741 | NW_020870788.1 | 138833 | C |
| A742 | NW_020870789.1 | 424358 | C |
| A743 | NW_020870796.1 | 190994 | G |
| A744 | NW_020870799.1 | 117724 | A |
| A745 | NW_020870800.1 | 615584 | C |
| A746 | NW_020870801.1 | 81823 | A |
| A747 | NW_020870805.1 | 127710 | T |
| A748 | NW_020870815.1 | 60551 | T |
| A749 | NW_020870816.1 | 86982 | C |
| A750 | NW_020870819.1 | 143332 | C |
| A751 | NW_020870820.1 | 394751 | T |
| A752 | NW_020870822.1 | 45912 | C |
| A753 | NW_020870823.1 | 498487 | A |
| A754 | NW_020870824.1 | 454950 | G |
| A755 | NW_020870830.1 | 93784 | T |
| A756 | NW_020870833.1 | 49888 | A |
| A757 | NW_020870834.1 | 142004 | G |
| A758 | NW_020870844.1 | 496843 | C |
| A759 | NW_020870846.1 | 252894 | T |
| A760 | NW_020870848.1 | 316954 | G |
| A761 | NW_020870849.1 | 365864 | T |
| A762 | NW_020870855.1 | 669693 | C |
| A763 | NW_020870859.1 | 736521 | A |
| A764 | NW_020870866.1 | 539344 | A |
| A765 | NW_020870873.1 | 93755 | C |
| A766 | NW_020870881.1 | 403210 | A |
| A767 | NW_020870886.1 | 61479 | T |
| A768 | NW_020870888.1 | 126022 | A |
| A769 | NW_020870896.1 | 843071 | A |
| A770 | NW_020870899.1 | 371515 | A |
| A771 | NW_020870901.1 | 726880 | A |
| A772 | NW_020870904.1 | 82149 | A |
| A773 | NW_020870905.1 | 8359 | A |
| A774 | NW_020870906.1 | 34374 | G |
| A775 | NW_020870907.1 | 160250 | C |
| A776 | NW_020870910.1 | 114269 | T |
| A777 | NW_020870911.1 | 1202067 | A |
| A778 | NW_020870914.1 | 226445 | C |
| A779 | NW_020870915.1 | 119073 | T |
| A780 | NW_020870916.1 | 201416 | A |
| A781 | NW_020870925.1 | 128551 | T |
| A782 | NW_020870928.1 | 1256299 | C |
| A783 | NW_020870929.1 | 344018 | C |
| A784 | NW_020870930.1 | 32068 | T |
| A785 | NW_020870937.1 | 201808 | A |
| A786 | NW_020870943.1 | 259344 | A |
| A787 | NW_020870947.1 | 381113 | C |
| A788 | NW_020870951.1 | 142935 | A |
| A789 | NW_020870952.1 | 268162 | A |
| A790 | NW_020870957.1 | 200803 | A |
| A791 | NW_020870969.1 | 423004 | A |
| A792 | NW_020870972.1 | 348449 | A |
| A793 | NW_020870977.1 | 7134 | T |
| A794 | NW_020870981.1 | 698781 | A |
| A795 | NW_020870983.1 | 149840 | T |
| A796 | NW_020870985.1 | 101089 | A |
| A797 | NW_020870988.1 | 1028385 | G |
| A798 | NW_020870989.1 | 372077 | C |
| A799 | NW_020870992.1 | 669198 | C |
| A800 | NW_020870999.1 | 840136 | A |
| A801 | NW_020871003.1 | 12395 | T |
| A802 | NW_020871005.1 | 352834 | T |
| A803 | NW_020871007.1 | 536053 | A |
| A804 | NW_020871008.1 | 406943 | A |
| A805 | NW_020871010.1 | 33070 | C |
| A806 | NW_020871014.1 | 341183 | T |
| A807 | NW_020871018.1 | 68100 | A |
| A808 | NW_020871022.1 | 161633 | A |
| A809 | NW_020871022.1 | 920136 | C |
| A810 | NW_020871023.1 | 232362 | C |
| A811 | NW_020871040.1 | 42967 | T |
| A812 | NW_020871040.1 | 1623245 | A |
| A813 | NW_020871044.1 | 198922 | C |
| A814 | NW_020871052.1 | 3275 | C |
| A815 | NW_020871056.1 | 184085 | T |
| A816 | NW_020871062.1 | 442063 | C |
| A817 | NW_020871071.1 | 60371 | C |
| A818 | NW_020871072.1 | 861708 | A |
| A819 | NW_020871078.1 | 489262 | A |
| A820 | NW_020871079.1 | 238412 | A |
| A821 | NW_020871081.1 | 136181 | T |
| A822 | NW_020871088.1 | 243252 | C |
| A823 | NW_020871090.1 | 590205 | T |
| A824 | NW_020871091.1 | 125020 | C |
| A825 | NW_020871094.1 | 1286443 | C |
| A826 | NW_020871095.1 | 32971 | G |
| A827 | NW_020871098.1 | 25541 | T |
| A828 | NW_020871100.1 | 135446 | T |
| A829 | NW_020871103.1 | 161383 | A |
| A830 | NW_020871109.1 | 20641 | T |
| A831 | NW_020871117.1 | 159404 | C |
| A832 | NW_020871125.1 | 179278 | G |
| A833 | NW_020871126.1 | 407792 | C |
| A834 | NW_020871133.1 | 22239 | T |
| A835 | NW_020871134.1 | 53044 | G |
| A836 | NW_020871135.1 | 194737 | A |
| A837 | NW_020871141.1 | 214649 | T |
| A838 | NW_020871164.1 | 513635 | A |
| A839 | NW_020871165.1 | 47635 | G |
| A840 | NW_020871170.1 | 164077 | G |
| A841 | NW_020871172.1 | 29669 | G |
| A842 | NW_020871172.1 | 280194 | T |
| A843 | NW_020871193.1 | 301731 | A |
| A844 | NW_020871204.1 | 18898 | G |
| A845 | NW_020871204.1 | 699554 | G |
| A846 | NW_020871214.1 | 56265 | A |
| A847 | NW_020871215.1 | 167934 | A |
| A848 | NW_020871223.1 | 89867 | T |
| A849 | NW_020871226.1 | 184460 | G |
| A850 | NW_020871232.1 | 812344 | T |
| A851 | NW_020871246.1 | 86514 | T |
| A852 | NW_020871249.1 | 645727 | T |
| A853 | NW_020871259.1 | 31134 | C |
| A854 | NW_020871259.1 | 548965 | T |
| A855 | NW_020871267.1 | 160387 | A |
| A856 | NW_020871270.1 | 15014 | A |
| A857 | NW_020871279.1 | 410484 | T |
| A858 | NW_020871293.1 | 73540 | T |
| A859 | NW_020871323.1 | 261651 | T |
| A860 | NW_020871329.1 | 32950 | A |
| A861 | NW_020871336.1 | 84187 | C |
| A862 | NW_020871339.1 | 129572 | A |
| A863 | NW_020871371.1 | 268164 | C |
| A864 | NW_020871391.1 | 321316 | G |
| A865 | NW_020871414.1 | 213183 | A |
| A866 | NW_020871420.1 | 70165 | A |
| A867 | NW_020871423.1 | 207593 | T |
| A868 | NW_020871428.1 | 607090 | C |
| A869 | NW_020871431.1 | 111207 | A |
| A870 | NW_020871451.1 | 48937 | G |
| A871 | NW_020871455.1 | 5884 | G |
| A872 | NW_020871463.1 | 131634 | A |
| A873 | NW_020871466.1 | 118754 | C |
| A874 | NW_020871470.1 | 1210883 | T |
| A875 | NW_020871485.1 | 235138 | C |
| A876 | NW_020871493.1 | 7663 | C |
| A877 | NW_020871497.1 | 62266 | A |
| A878 | NW_020871504.1 | 167741 | A |
| A879 | NW_020871507.1 | 103632 | A |
| A880 | NW_020871546.1 | 616652 | G |
| A881 | NW_020871553.1 | 283260 | A |
| A882 | NW_020871567.1 | 951158 | C |
| A883 | NW_020871575.1 | 132550 | A |
| A884 | NW_020871579.1 | 277779 | C |
| A885 | NW_020871588.1 | 1410174 | A |
| A886 | NW_020871610.1 | 445228 | C |
| A887 | NW_020871618.1 | 65888 | C |
| A888 | NW_020871622.1 | 208046 | G |
| A889 | NW_020871624.1 | 338162 | G |
| A890 | NW_020871628.1 | 24099 | A |
| A891 | NW_020871636.1 | 789835 | T |
| A892 | NW_020871663.1 | 864899 | C |
| A893 | NW_020871694.1 | 157672 | A |
| A894 | NW_020871694.1 | 598562 | T |
| A895 | NW_020871708.1 | 315056 | A |
| A896 | NW_020871718.1 | 17932 | C |
| A897 | NW_020871724.1 | 548921 | G |
| A898 | NW_020871725.1 | 39404 | A |
| A899 | NW_020871728.1 | 11563 | C |
| A900 | NW_020871737.1 | 299780 | T |
| A901 | NW_020871744.1 | 231757 | G |
| A902 | NW_020871810.1 | 222347 | A |
| A903 | NW_020871821.1 | 125920 | A |
| A904 | NW_020871827.1 | 210865 | A |
| A905 | NW_020871828.1 | 71490 | A |
| A906 | NW_020871828.1 | 206545 | T |
| A907 | NW_020871838.1 | 86248 | G |
| A908 | NW_020871841.1 | 714139 | C |
| A909 | NW_020871866.1 | 83780 | A |
| A910 | NW_020871871.1 | 54121 | G |
| A911 | NW_020871916.1 | 128659 | C |
| A912 | NW_020871918.1 | 96448 | C |
| A913 | NW_020871922.1 | 224337 | A |
| A914 | NW_020871923.1 | 517756 | C |
| A915 | NW_020871926.1 | 594840 | A |
| A916 | NW_020871936.1 | 52614 | G |
| A917 | NW_020871947.1 | 1458 | C |
| A918 | NW_020871947.1 | 802061 | G |
| A919 | NW_020871997.1 | 398814 | G |
| A920 | NW_020871998.1 | 174553 | A |
| A921 | NW_020871999.1 | 162163 | G |
| A922 | NW_020872010.1 | 19152 | A |
| A923 | NW_020872016.1 | 8103 | A |
| A924 | NW_020872066.1 | 302570 | A |
| A925 | NW_020872068.1 | 296227 | A |
| A926 | NW_020872073.1 | 181498 | T |
| A927 | NW_020872074.1 | 331440 | T |
| A928 | NW_020872085.1 | 233836 | G |
| A929 | NW_020872098.1 | 318997 | A |
| A930 | NW_020872101.1 | 164968 | A |
| A931 | NW_020872103.1 | 17541 | T |
| A932 | NW_020872111.1 | 168132 | C |
| A933 | NW_020872140.1 | 727925 | C |
| A934 | NW_020872143.1 | 501370 | C |
| A935 | NW_020872149.1 | 52221 | G |
| A936 | NW_020872174.1 | 16383 | C |
| A937 | NW_020872191.1 | 495543 | A |
| A938 | NW_020872203.1 | 281305 | A |
| A939 | NW_020872231.1 | 2844 | T |
| A940 | NW_020872232.1 | 86836 | C |
| A941 | NW_020872236.1 | 20858 | A |
| A942 | NW_020872248.1 | 62651 | T |
| A943 | NW_020872253.1 | 265725 | T |
| A944 | NW_020872260.1 | 630806 | C |
| A945 | NW_020872284.1 | 959702 | G |
| A946 | NW_020872300.1 | 198217 | G |
| A947 | NW_020872305.1 | 9972 | A |
| A948 | NW_020872307.1 | 126478 | A |
| A949 | NW_020872317.1 | 460853 | G |
| A950 | NW_020872325.1 | 451258 | G |
| A951 | NW_020872328.1 | 134269 | G |
| A952 | NW_020872336.1 | 73961 | T |
| A953 | NW_020872340.1 | 10631 | G |
| A954 | NW_020872366.1 | 310484 | A |
| A955 | NW_020872381.1 | 42586 | C |
| A956 | NW_020872385.1 | 479620 | C |
| A957 | NW_020872386.1 | 48885 | A |
| A958 | NW_020872393.1 | 61516 | A |
| A959 | NW_020872398.1 | 104539 | C |
| A960 | NW_020872401.1 | 95183 | C |
| A961 | NW_020872404.1 | 320548 | T |
| A962 | NW_020872407.1 | 103771 | C |
| A963 | NW_020872417.1 | 182317 | T |
| A964 | NW_020872420.1 | 1312303 | A |
| A965 | NW_020872422.1 | 186883 | C |
| A966 | NW_020872423.1 | 330780 | A |
| A967 | NW_020872424.1 | 213784 | T |
| A968 | NW_020872428.1 | 351239 | A |
| A969 | NW_020872431.1 | 357026 | T |
| A970 | NW_020872432.1 | 1097678 | G |
| A971 | NW_020872433.1 | 746864 | C |
| A972 | NW_020872437.1 | 562823 | C |
| A973 | NW_020872443.1 | 606210 | C |
| A974 | NW_020872445.1 | 199229 | T |
| A975 | NW_020872449.1 | 887334 | G |
| A976 | NW_020872450.1 | 639457 | A |
| A977 | NW_020872452.1 | 17117 | G |
| A978 | NW_020872454.1 | 508332 | T |
| A979 | NW_020872455.1 | 200462 | T |
| A980 | NW_020872463.1 | 26897 | C |
| A981 | NW_020872468.1 | 241027 | C |
| A982 | NW_020872475.1 | 181198 | A |
| A983 | NW_020872479.1 | 301337 | C |
| A984 | NW_020872482.1 | 209916 | G |
| A985 | NW_020872484.1 | 134422 | G |
| A986 | NW_020872485.1 | 551973 | C |
| A987 | NW_020872489.1 | 642409 | G |
| A988 | NW_020872494.1 | 330157 | A |
| A989 | NW_020872495.1 | 150710 | G |
| A990 | NW_020872504.1 | 341724 | T |
| A991 | NW_020872508.1 | 86300 | T |
| A992 | NW_020872512.1 | 46095 | T |
| A993 | NW_020872514.1 | 179357 | G |
| A994 | NW_020872521.1 | 388857 | T |
| A995 | NW_020872537.1 | 122521 | T |
| A996 | NW_020872540.1 | 44079 | T |
| A997 | NW_020872541.1 | 594195 | A |
| A998 | NW_020872545.1 | 125318 | G |
| A999 | NW_020872547.1 | 441451 | C |
| A1000 | NW_020872554.1 | 120989 | A |
| A1001 | NW_020872556.1 | 133330 | A |
| A1002 | NW_020872557.1 | 59929 | T |
| A1003 | NW_020872559.1 | 647842 | T |
| A1004 | NW_020872561.1 | 1094981 | C |
| A1005 | NW_020872567.1 | 278023 | C |
| A1006 | NW_020872570.1 | 176644 | C |
| A1007 | NW_020872571.1 | 41392 | A |
| A1008 | NW_020872573.1 | 513152 | C |
| A1009 | NW_020872576.1 | 65500 | G |
| A1010 | NW_020872577.1 | 1558 | G |
| A1011 | NW_020872597.1 | 104829 | C |
| A1012 | NW_020872599.1 | 529727 | G |
| A1013 | NW_020872601.1 | 199574 | A |
| A1014 | NW_020872607.1 | 564921 | T |
| A1015 | NW_020872608.1 | 845613 | A |
| A1016 | NW_020872610.1 | 597897 | A |
| A1017 | NW_020872620.1 | 39757 | T |
| A1018 | NW_020872622.1 | 990808 | A |
| A1019 | NW_020872632.1 | 402945 | T |
| A1020 | NW_020872634.1 | 380697 | A |
| A1021 | NW_020872640.1 | 166505 | A |
| A1022 | NW_020872642.1 | 587186 | A |
| A1023 | NW_020872655.1 | 169299 | A |
| A1024 | NW_020872656.1 | 8572 | G |
| A1025 | NW_020872667.1 | 148934 | G |
| A1026 | NW_020872670.1 | 185804 | C |
| A1027 | NW_020872671.1 | 101534 | G |
| A1028 | NW_020872676.1 | 9790 | T |
| A1029 | NW_020872677.1 | 127841 | C |
| A1030 | NW_020872686.1 | 459876 | C |
| A1031 | NW_020872687.1 | 58139 | A |
| A1032 | NW_020872693.1 | 420144 | G |
| A1033 | NW_020872696.1 | 961694 | G |
| A1034 | NW_020872697.1 | 109321 | A |
| A1035 | NW_020872701.1 | 158099 | A |
| A1036 | NW_020872701.1 | 1489216 | A |
| A1037 | NW_020872704.1 | 155165 | C |
| A1038 | NW_020872706.1 | 177621 | C |
| A1039 | NW_020872708.1 | 281903 | G |
| A1040 | NW_020872710.1 | 113125 | A |
| A1041 | NW_020872716.1 | 367373 | T |
| A1042 | NW_020872717.1 | 414534 | T |
| A1043 | NW_020872719.1 | 1271875 | A |
| A1044 | NW_020872720.1 | 190774 | A |
| A1045 | NW_020872722.1 | 361970 | C |
| A1046 | NW_020872723.1 | 195236 | A |
| A1047 | NW_020872726.1 | 836365 | C |
| A1048 | NW_020872728.1 | 303057 | T |
| A1049 | NW_020872734.1 | 968024 | A |
| A1050 | NW_020872735.1 | 351612 | C |
| A1051 | NW_020872737.1 | 2198 | C |
| A1052 | NW_020872741.1 | 423351 | G |
| A1053 | NW_020872742.1 | 460233 | A |
| A1054 | NW_020872743.1 | 57166 | T |
| A1055 | NW_020872745.1 | 33851 | G |
| A1056 | NW_020872748.1 | 94919 | T |
| A1057 | NW_020872749.1 | 342867 | T |
| A1058 | NW_020872755.1 | 237326 | A |
| A1059 | NW_020872761.1 | 399245 | C |
| A1060 | NW_020872762.1 | 737201 | C |
| A1061 | NW_020872766.1 | 1445301 | T |
| A1062 | NW_020872772.1 | 716456 | G |
| A1063 | NW_020872787.1 | 321840 | A |
| A1064 | NW_020872788.1 | 1175690 | A |
| A1065 | NW_020872800.1 | 31240 | C |
| A1066 | NW_020872804.1 | 433499 | T |
| A1067 | NW_020872806.1 | 62981 | A |
| A1068 | NW_020872808.1 | 631383 | A |
| A1069 | NW_020872814.1 | 175647 | C |
| A1070 | NW_020872817.1 | 167185 | T |
| A1071 | NW_020872820.1 | 21802 | A |
| A1072 | NW_020872825.1 | 298620 | A |
| A1073 | NW_020872826.1 | 162193 | G |
| A1074 | NW_020872833.1 | 83602 | C |
| A1075 | NW_020872837.1 | 7777 | T |
| A1076 | NW_020872850.1 | 464375 | C |
| A1077 | NW_020872851.1 | 587485 | G |
| A1078 | NW_020872855.1 | 124988 | G |
| A1079 | NW_020872859.1 | 315169 | A |
| A1080 | NW_020872860.1 | 64784 | T |
| A1081 | NW_020872872.1 | 21216 | C |
| A1082 | NW_020872873.1 | 460793 | C |
| A1083 | NW_020872875.1 | 292067 | A |
| A1084 | NW_020872876.1 | 242453 | C |
| A1085 | NW_020872878.1 | 132949 | A |
| A1086 | NW_020872879.1 | 232082 | T |
| A1087 | NW_020872882.1 | 709105 | A |
| A1088 | NW_020872894.1 | 488897 | C |
| A1089 | NW_020872896.1 | 2277 | A |
| A1090 | NW_020872906.1 | 3701 | C |
| A1091 | NW_020872907.1 | 1217777 | A |
| A1092 | NW_020872908.1 | 28747 | A |
| A1093 | NW_020872909.1 | 19217 | A |
| A1094 | NW_020872911.1 | 710111 | G |
| A1095 | NW_020872915.1 | 1377934 | A |
| A1096 | NW_020872922.1 | 259906 | T |
| A1097 | NW_020872926.1 | 3062 | G |
| A1098 | NW_020872930.1 | 934371 | G |
| A1099 | NW_020872942.1 | 180619 | T |
| A1100 | NW_020872943.1 | 301600 | G |
| A1101 | NW_020872943.1 | 571615 | A |
| A1102 | NW_020872945.1 | 27348 | C |
| A1103 | NW_020872948.1 | 56686 | C |
| A1104 | NW_020872955.1 | 320871 | G |
| A1105 | NW_020872956.1 | 643111 | A |
| A1106 | NW_020872958.1 | 627495 | T |
| A1107 | NW_020872964.1 | 62895 | A |
| A1108 | NW_020872966.1 | 504876 | A |
| A1109 | NW_020868585.1 | 48666 | G |
| A1110 | NW_020868711.1 | 1597 | C |
| A1111 | NW_020868724.1 | 18739 | A |
| A1112 | NW_020870268.1 | 30442 | T |
| A1113 | NW_020870909.1 | 36878 | T |
| A1114 | NW_020871119.1 | 4978 | T |
| A1115 | NW_020871190.1 | 9219 | A |
| A1116 | NW_020871592.1 | 55702 | A |
| A1117 | NW_020871677.1 | 5985 | T |
| A1118 | NW_020871704.1 | 4034 | G |
| A1119 | NW_020871851.1 | 665 | A |
| A1120 | NW_020872186.1 | 24415 | C |
| A1121 | NW_020872220.1 | 455 | A |
| A1122 | NW_020872295.1 | 19691 | A |
| A1123 | NW_020872351.1 | 68606 | A |
| A1124 | NW_020872793.1 | 70352 | G |
| A1125 | NW_020872803.1 | 35946 | T |

The present invention employs an SNP site combination for parentage identification, eliminating the need for early separate rearing of different families and enabling early mixed rearing of family individuals. This approach reduces common environmental effects arising from early separate family rearing and thereby improves the accuracy of Litopenaeus vannamei family mixed breeding trait evaluation. The SNP site combination enables accurate calculation of kinship between individuals and yields reliable pedigrees without the risk of detachment, misplacement, or human reading errors associated with physical tagging. Using the SNP site combination to identify family membership permits precise assessment of family-level survival and growth traits. These SNP (Single Nucleotide Polymorphism) molecular markers are characterized by large numbers, broad genomic distribution, and suitability for high-throughput detection. As used herein, “ID” means identification, site identifier; “Scaffold” refers to the genomic scaffold; “Position” denotes physical coordinate on the scaffold; and “Ref” denotes the reference genome base at the site. The Litopenaeus vannamei reference genome sequence used in the present invention is available at: https://www.ncbi.nlm.nih.gov/datasets/taxonomy/6689/.

The present invention further provides a molecular probe for use in Litopenaeus vannamei family mixed breeding trait evaluation, wherein the molecular probe is a single-stranded DNA probe. Each probe comprises 1 to 4 distinct nucleotide sequences designed with differential characteristics based on the foregoing SNP sites. Each nucleotide sequence is 110 base pairs (bp) in length and is designed to provide averaging 55 bp of upstream and 55 bp of downstream sequence coverage surrounding the SNP site. As used herein, “DNA” refers to deoxyribonucleic acid and “bp” represents base pairs.

The molecular probe is configured to detect the Litopenaeus vannamei SNP site combination, and the probe set is capable of targeted capture of 1,125 target regions. Based on the designed nucleotide sequences, single-stranded nucleotide synthesis is performed with a synthesis length of 110 bp. DNA nucleotide sequences modified with biotin at the 5′ end serve as molecular probes. The synthesized molecular probes are combined in equimolar mass using a mixed EDTA and Tris-HCL solution to prepare the probe mixture. EDTA (ethylenediaminetetraacetic acid) has the chemical formula C₁₀H₁₆N₂O₈; Tris-HCL (Tris (hydroxymethyl) Aminomethane Hydrochloride) has the molecular formula C₄H₁₂ClNO₃.

The present invention provides a kit for application in Litopenaeus vannamei family mixed breeding trait evaluation, the kit comprising a probe mixture and hybridization capture reagents, wherein the probe mixture is prepared from the molecular probes described above for application in Litopenaeus vannamei family mixed breeding trait evaluation.

The kit for application in Litopenaeus vannamei family mixed breeding trait evaluation comprises the probe mixture and hybridization capture reagents, which are independently packaged. The probe mixture is prepared from the molecular probes, and the hybridization capture reagents typically include Block I, Block II, 2×Hyb Buffer, Hyb Buffer Enhancer, Primer Mix, PCR Master Mix, 2× Beads Wash Buffer, 10× Wash Buffer I, 10× Wash Buffer II, 10× Wash Buffer III, 10× Strigent Wash Buffer, DNA Probe Beads, and DNA Clean Beads. These hybridization capture reagents are preferably sourced from Shijiazhuang Boruidi Biotechnology Co., Ltd. The kit is low-cost and provides high accuracy.

As a preferred embodiment, an application of the kit in genotype information identification includes the following steps:
1) Fragment the DNA of the Litopenaeus vannamei sample, perform end repair and adapter ligation, purify to obtain a sequencing library, then perform library amplification and library purification to obtain purified library DNA;
2) Add the probe mixture and hybridization capture reagents from the kit to the purified library DNA obtained in step 1), perform hybridization capture and elution to remove non-target DNA fragments, thereby enriching target DNA fragments that include the SNP sites, then amplify and purify the target DNA to construct a targeted sequencing library;
3) Assess the targeted sequencing library obtained in step 2) for qualification, perform DNA sequencing using second-generation sequencing technology after the targeted sequencing library passes quality assessment, and compare the sequencing reads to the Litopenaeus vannamei reference genome sequence to obtain the genotype information of the tested Litopenaeus vannamei.

The kit applied in Litopenaeus vannamei family mixed breeding trait evaluation is used to identify Litopenaeus vannamei genotype information by a targeted capture sequencing genotyping method. The method employs the molecular probes of the present invention to directionally capture target region segments, namely DNA fragments containing target sites, producing target DNA fragments. Following DNA sequencing and alignment to the Litopenaeus vannamei reference genome sequence, the genotype information of the tested Litopenaeus vannamei is obtained. This genotyping method is simple to operate, cost-effective, consistent across different sequencing batches, flexible in site selection, and readily upgradeable.

As a preferred embodiment, an application of the kit in Litopenaeus vannamei family mixed breeding trait evaluation includes the following steps:

### S1 Preparation

1) Select shrimp larvae from different families, with the number of families not less than 10;
2) Take the paternal, maternal, and sibling individuals of the shrimp larvae from step 1) as target individuals, and determine the genotype information of the target individuals using the steps set forth in the kit's application for genotype information identification;

### S2 Mixed Breeding

3) From each family in step 1), randomly select 30-50 shrimp larvae and evenly distribute them into multiple parallel breeding pools for mixed rearing, maintain water temperature not lower than 23° C., and rear for 60-90 days to obtain test individuals;

### S3 Trait Evaluation

4) Count the number of test individuals in each breeding pool, collect the test individuals, and measure their body weight;
5) Determine the genotype information of the test individuals obtained in step 4) using the steps in the kit's application for genotype information identification;
6) Reconstruct pedigree relationships by combining the genotype information of the tested individuals from step 5) with the genotype information of the target individuals from step 2), thereby obtaining family assignment for the test individuals;
7) Based on the family assignment from step 6), calculate survival counts and body weight for the test individuals from different families to obtaining the survival and growth traits for the Litopenaeus vannamei family mixed breeding groups.

The kit applied in Litopenaeus vannamei family mixed breeding trait evaluation is used to assess Litopenaeus vannamei family mixed breeding traits. It is a method for evaluating Litopenaeus vannamei family mixed breeding traits, specifically survival and growth traits, by means of SNP site combination-based parentage identification and targeted capture genotyping. This method obviates the need for separate early-family rearing and replaces traditional VIE marking, enabling early mixed rearing of family individuals. Consequently, common environmental effects caused by separate early-family rearing are reduced and the accuracy of Litopenaeus vannamei family mixed breeding trait evaluation is improved. Compared with traditional VIE marker identification, the SNP site combination of the present invention permits accurate kinship calculation and pedigree reconstruction without risks of marker detachment, misplacement, or human reading errors. The evaluation method for Litopenaeus vannamei family mixed breeding traits is low-cost and enables effective assessment of survival and growth traits in Litopenaeus vannamei mixed-family breeding.

As a preferred embodiment, in step 1) the shrimp larvae are at the P15-P40 stage. The present invention selects shrimp larvae in the P15-P40 stage for mixed breeding testing, where P15-P40 refers to 15-40 days after metamorphosis of the shrimp larvae. Larvae at this stage are 15-40 days old and in good growth condition. Mixed breeding is normally conducted for 60-90 days, at the end of which test individuals typically reach a body weight close to 15 g; the exact mixed breeding duration is determined by comprehensive consideration of the rearing environment and survival rate.

As a preferred embodiment, in step 2), sibling individuals used for genotype identification are at P45 and above. The present invention selects sibling individuals at P45 and above for larvae genotype identification, where P45 refers to 45 days after metamorphosis of the shrimp larvae. These larger sibling individuals facilitate DNA extraction and fully ensure the quality of genotype identification.

As a preferred embodiment, in step 2), during the process of identifying target individuals' genotype information: if genotyping of both parents is successful, select 1-3 sibling individuals from the family for genotyping; if genotyping of one parent is unsuccessful, select 2-4 sibling individuals for genotyping; if genotyping of both parents is unsuccessful, select 3-5 sibling individuals for genotyping. This tiered genotyping strategies for target individuals ensures an adequate number of sampled siblings to meet subsequent requirements for reconstructing test individuals' family information.

As a preferred embodiment, in step 4), the stocking density of larvae from each family in each breeding pool is 100-300 individuals/m³. Typically 2-3 parallel pools are established, and the number of larvae taken from each family is adjusted according to the chosen breeding density and the total number of families.

As a preferred embodiment, in step 6): when genotype information for both parents is available among the target individuals, pedigree reconstruction is performed using the genotype information of the test individuals together with the parents' genotype information of the target individuals, thereby assigning family membership to the test individuals; when genotype information for both parents is absent, clustering analysis is performed using the genotype information of test individuals and the sibling individuals within the target individuals, and family assignments for test individuals are inferred based on the known family identities of the sibling individuals within the target individuals. The present invention comprehensively addresses family assignment, enabling accurate kinship calculation between individuals and precise discrimination of Litopenaeus vannamei families.

Compared to the prior art, the beneficial effects of the present invention include: using SNP site combinations for parentage identification eliminates the need for separate family rearing and replaces traditional VIE marking, enabling early mixed breeding of different family individuals and thereby reducing common environmental effects from early single-family rearing and improving the accuracy of Litopenaeus vannamei family mixed breeding trait evaluation. Moreover, relative to traditional VIE marker family identification, the SNP site combination allows precise kinship calculation among individuals and unamibiguous differentiation of Litopenaeus vannamei families without risks of individual marker detachment, misplacement, or human reading errors, yielding reliable pedigrees. The Litopenaeus vannamei family mixed breeding trait evaluation method is low-cost and enables effective assessment of survival and growth traits in mixed breeding.

### DETAILED DESCRIPTION OF THE DRAWINGS

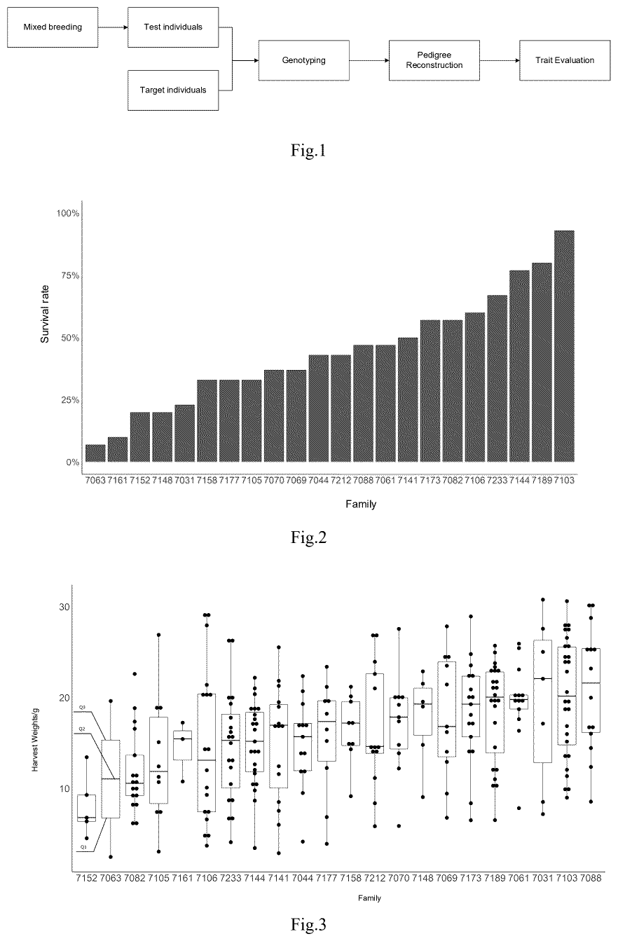

FIG. 1 is a flowchart of the Litopenaeus vannamei family mixed breeding trait evaluation method provided by the present invention.

FIG. 2 is a bar graph showing the survival rates of test individuals from Embodiment 2.

FIG. 3 is a Box plot showing the body weights of test individuals from Embodiment 2.

In FIG. 3:
black dots represent individual harvest weights;
the box shows the interquartile range of individual harvest weight data, where the lower box edge is the first quartile (Q1), the upper box edge represents the third quartile (Q3), the middle line represents the median (Q2), and the box length represents the interquartile range (IQR).

### DETAILED DESCRIPTION

The technical solution of the invention will be described clearly and completely in conjunction with specific embodiment examples. Obviously, the described embodiments are merely part of the invention's embodiments, not all embodiments. Based on the embodiments in this invention, all other embodiments obtained by those skilled in the art without creative labor are within the scope of the invention's protection.

### Best Embodiment of the Invention

An application of SNP site combinations in Litopenaeus vannamei family mixed breeding trait evaluation, wherein the SNP site combination includes 1,125 SNP sites. The physical positions of the SNP sites are determined by alignment to the Litopenaeus vannamei reference genome sequence, and the SNP site information is provided in Table 1, in which ID denotes the site number, Scaffold denotes the scaffold name of the Litopenaeus vannamei reference genome containing the site, Position denotes the physical coordinate on the scaffold, and Ref denotes the reference genome base at the site.

A molecular probe applied in Litopenaeus vannamei family mixed breeding trait evaluation, wherein the molecular probe is a single-stranded DNA probe designed based on the SNP sites of the above-described Litopenaeus vannamei SNP site combination. Each probe comprises 1~4 distinct nucleotide sequences, each nucleotide sequence being 110 bp in length and covering, on average, 55 bp upstream and 55 bp downstream of the SNP site.

A kit applied in Litopenaeus vannamei family mixed breeding trait evaluation, wherein the kit comprises a probe mixing solution and a hybridization capture reagent, and the probe mixing solution is prepared by the molecular probe applied in Litopenaeus vannamei family mixed breeding trait evaluation as described above.

Preferably, the application of the kit in genotype information identification comprises the following steps:
1) Fragment the DNA of the Litopenaeus vannamei sample, perform end repair and adapter ligation, purify to obtain a sequencing library, then perform library amplification and library purification to obtain purified library DNA;
2) Add the probe mixture and hybridization capture reagents from the kit to the purified library DNA obtained in step 1), perform hybridization capture and elution to remove non-target DNA fragments, thereby enriching target DNA fragments that include the SNP sites, then amplify and purify the target DNA to construct a targeted sequencing library;
3) Assess the targeted sequencing library obtained in step 2) for qualification, perform DNA sequencing using second-generation sequencing technology, and compare the sequencing reads to the Litopenaeus vannamei reference genome sequence to obtain the genotype information of the tested Litopenaeus vannamei.

Preferably, the application of the kit in Litopenaeus vannamei family mixed breeding trait evaluation comprises the following steps:

### S1 Preparation

1) Select shrimp larvae from different families, with the number of families not less than 10;
2) Take the paternal, maternal, and sibling individuals of the shrimp larvae from step 1) as target individuals, and determine the genotype information of the target individuals using the steps set forth in the kit's application for genotype information identification;

### S2 Mixed Breeding

3) From each family in step 1), randomly select 30-50 shrimp larvae and evenly distribute them into multiple parallel breeding pools for mixed rearing, maintain water temperature not lower than 23° C., and rear for 60-90 days to obtain test individuals;

### S3 Trait Evaluation

4) Count the number of test individuals in each breeding pool, measure their body weight;
5) Use the kit's steps to identify the genotype information of the test individuals obtained in step 4);
6) Reconstruct the pedigree using the test individuals' genotype information from step 5) and target individuals' genotype information from step 2) to obtain the test individuals' family information;
7) Based on the family information from step 6), count survival numbers and body weights of test individuals from different families to obtain survival and growth traits of Litopenaeus vannamei family mixed breeding Count the number of test individuals in each breeding pool, collect the test individuals, and measure their body weight;
Preferably, in step 1), the shrimp larvae are at the P15-P40 stage.

Preferably, in step 2), sibling individuals used for genotype identification are at P45 and above.

Preferably, in step 2), during the process of identifying target individuals' genotype information: if genotyping of both parents is successful, select 1-3 sibling individuals from the family for genotyping; if genotyping of one parent is unsuccessful, select 2-4 sibling individuals for genotyping; if genotyping of both parents is unsuccessful, select 3-5 sibling individuals for genotyping.

Preferably, in step 3), the stocking density of larvae from each family in each breeding pool is 100-300 individuals/m³.

Preferably, in step 6): when genotype information for both parents is available among the target individuals, pedigree reconstruction is performed using the genotype information of the test individuals together with the parents' genotype information of the target individuals; when genotype information for both parents is absent, clustering analysis is performed using the genotype information of test individuals and the sibling individuals within the target individuals, and family assignments for test individuals are inferred based on the known family identities of the sibling individuals within the target individuals.

### Embodiment 1

A kit applied in Litopenaeus vannamei family mixed breeding trait evaluation, with its application in genotype information identification, comprises the following steps:

1) Extraction and Detection of Litopenaeus vannamei Genomic DNA

Select muscle, swimming foot, or antenna tissue from Litopenaeus vannamei and extract DNA using a high-throughput DNA kit; perform two assessments on the extracted DNA: (a) evaluate DNA purity and integrity by 1% agarose gel electrophoresis, and (b) accurately quantify DNA concentration using a Qubit fluorometer.

### 2) DNA Library Construction

Use DNA samples that meet the quantitative quality criteria, and randomly fragment using ultrasonic shearing, controlling the fragment peak at 200-300 bp. Perform end repair and add A-tail

Ligate A-tailed DNA fragments to sequencing adapters using ligase, then purify and perform size selection with carboxyl-modified magnetic beads, retaining ligation products with insert sizes of 200-300 bp.

Add sequencing primers containing barcode and a high-fidelity PCR mix to the ligation products and perform PCR amplification; use different barcodes to distinguish different DNA samples. Purify the amplified products with carboxyl magnetic beads to obtain purified library DNA.

### 3) Targeted Sequencing Library Construction

Take 500 ng purified library DNA, freeze-dry, add the probe mixing solution and hybridization capture reagent of the kit, denature and incubate at 65° C. for 2 h to complete the hybridization capture reaction.

Wash the hybridization capture products with washing solution to remove non-target DNA fragments and recover target DNA fragments.

Amplify and purify the recovered target DNA fragments to construct the targeted sequencing library.

### 4) DNA Sequencing

Perform preliminary quantification after the targeted sequencing library construction using a Qubit 2.0 fluorescence quantifier, then use qPCR

(Quantitative Real-time Polymerase Chain Reaction) to accurately quantify the effective concentration of the targeted sequencing library to ensure library quality.

After the targeted sequencing library passes quality assessment, perform DNA sequencing using second-generation sequencing technology.

### 5) Genotype Analysis

Perform data quality assessment (remove adapters and low-quality reads), align the sequencing data to the Litopenaeus vannamei reference genome sequence, and detect variants to obtain the genotype information of the tested Litopenaeus vannamei.

### Embodiment 2

Referring to FIG. 1, a kit applied in Litopenaeus vannamei family mixed breeding trait evaluation, with its application in Litopenaeus vannamei family mixed breeding trait evaluation, comprises the following steps:

### S1 Preparation Work

1) In July 2023, establish a G6 generation (bred to the sixth generation) core breeding population of Litopenaeus vannamei and select 22 families from this population;
2) From larvae families, collect parent, maternal, and sibling individuals, and from each family select 5 individuals with known family information as target individuals for subsequent identification; determine the genotype information of the target individuals using the method in the Embodiment One. Across the 22 families, genotype information was successfully obtained for 74 sibling individuals and 15 maternal individuals;

### S2 Mixed Breeding

3) From each family identified in step 1), randomly select 30 larvae individuals at the P15 growth stage and distribute them evenly into 3 parallel breeding pools, maintain a breeding density of 110 individuals/m³, and perform mixed breeding at a water temperature of 28° C. for 90 days to obtain the test individuals;

### S3 Trait Evaluation

4) Count the number of test individuals in each breeding pool and measure their body weight;
5) Identify genotype information of test individuals from step 4) using the method in the Embodiment One;
6) Obtain 1K SNP genotype information from 292 individuals and use the sequoia package in R language to reconstruct pedigree and perform sibling clustering, incorporating the target individuals' genotype information from step 2);
7) Using the family assignment from step 6), count survival numbers and body weights of test individuals from different families to obtain survival and growth traits of Litopenaeus vannamei family mixed breeding;
From FIG. 2, the present invention clearly distinguishes survival rates between families. From FIG. 3, the present invention clearly distinguishes harvest body weights between families.

Therefore, compared with the prior art, the beneficial effects of the present invention are: the present invention employs SNP site combinations for parentage identification, eliminating the need for separate rearing of different families and replacing traditional VIE marking; it enables early mixed breeding among families, effectively reducing common environmental effects associated with single-family rearing and improving the accuracy of Litopenaeus vannamei family mixed breeding trait evaluation. Compared with traditional VIE marker identification, the SNP site combination in the present invention can accurately calculate kinship between individuals and precisely distinguish Litopenaeus vannamei families, avoiding marker loss, misplacement, and human identification errors, thereby yielding an accurate pedigree. The Litopenaeus vannamei mixed breeding trait evaluation method of the present invention is low-cost and enables effective assessment of survival and growth traits in family mixed breeding.

The above description is merely the preferred embodiment of the invention and should not be used to limit the invention. Any modifications, equivalent substitutions, or improvements made within the spirit and principles of the invention should be included within its protection scope.

1. An application of an SNP site combination of Litopenaeus vannamei for use in family mixed breeding trait evaluation, comprising an SNP site combination comprising 1,125 SNP sites, wherein the physical positions of the SNP sites are determined by comparison to the Litopenaeus vannamei reference genome sequence, and wherein information for the SNP sites is shown in Table 1, wherein “ID” is the site identifier, “Scaffold” is the name of the scaffold in the Litopenaeus vannamei reference genome on which the site is located, “Position” is the physical coordinate of the site on the scaffold, and “Ref” is the reference genome base at the site for Litopenaeus vannamei;
TABLE 1
Information for the SNP Sites

| ID | Scaffold | Position | Ref |
|---|---|---|---|
| A1 | NW_020868289.1 | 207791 | G |
| A2 | NW_020868292.1 | 51443 | C |
| A3 | NW_020868293.1 | 255982 | G |
| A4 | NW_020868301.1 | 214864 | T |
| A5 | NW_020868304.1 | 155318 | G |
| A6 | NW_020868305.1 | 720780 | C |
| A7 | NW_020868306.1 | 207725 | A |
| A8 | NW_020868307.1 | 582033 | G |
| A9 | NW_020868311.1 | 501920 | C |
| A10 | NW_020868316.1 | 42724 | T |
| A11 | NW_020868318.1 | 292168 | A |
| A12 | NW_020868324.1 | 94414 | C |
| A13 | NW_020868328.1 | 310383 | T |
| A14 | NW_020868334.1 | 184665 | A |
| A15 | NW_020868335.1 | 210210 | C |
| A16 | NW_020868337.1 | 214071 | G |
| A17 | NW_020868341.1 | 493235 | A |
| A18 | NW_020868344.1 | 222217 | T |
| A19 | NW_020868345.1 | 326144 | C |
| A20 | NW_020868351.1 | 136314 | T |
| A21 | NW_020868353.1 | 384502 | T |
| A22 | NW_020868357.1 | 682725 | T |
| A23 | NW_020868360.1 | 335685 | C |
| A24 | NW_020868364.1 | 251991 | G |
| A25 | NW_020868366.1 | 81392 | C |
| A26 | NW_020868369.1 | 13259 | G |
| A27 | NW_020868377.1 | 169009 | A |
| A28 | NW_020868379.1 | 204340 | C |
| A29 | NW_020868386.1 | 33176 | A |
| A30 | NW_020868390.1 | 130449 | A |
| A31 | NW_020868393.1 | 310814 | C |
| A32 | NW_020868394.1 | 328073 | T |
| A33 | NW_020868397.1 | 96282 | G |
| A34 | NW_020868406.1 | 3844 | G |
| A35 | NW_020868415.1 | 1652844 | C |
| A36 | NW_020868416.1 | 212490 | G |
| A37 | NW_020868429.1 | 333337 | C |
| A38 | NW_020868431.1 | 98599 | C |
| A39 | NW_020868436.1 | 216158 | C |
| A40 | NW_020868439.1 | 337192 | T |
| A41 | NW_020868441.1 | 655324 | T |
| A42 | NW_020868444.1 | 944654 | T |
| A43 | NW_020868450.1 | 175085 | G |
| A44 | NW_020868453.1 | 712720 | T |
| A45 | NW_020868455.1 | 160936 | C |
| A46 | NW_020868456.1 | 12986 | G |
| A47 | NW_020868458.1 | 795878 | C |
| A48 | NW_020868461.1 | 49289 | C |
| A49 | NW_020868467.1 | 243436 | G |
| A50 | NW_020868468.1 | 994083 | A |
| A51 | NW_020868479.1 | 38677 | T |
| A52 | NW_020868481.1 | 510733 | C |
| A53 | NW_020868484.1 | 19731 | A |
| A54 | NW_020868486.1 | 181933 | C |
| A55 | NW_020868495.1 | 169988 | T |
| A56 | NW_020868506.1 | 263825 | T |
| A57 | NW_020868506.1 | 1054292 | T |
| A58 | NW_020868512.1 | 955288 | A |
| A59 | NW_020868523.1 | 464246 | A |
| A60 | NW_020868534.1 | 22047 | T |
| A61 | NW_020868537.1 | 421538 | A |
| A62 | NW_020868538.1 | 321994 | C |
| A63 | NW_020868539.1 | 201636 | G |
| A64 | NW_020868542.1 | 427985 | G |
| A65 | NW_020868547.1 | 275291 | G |
| A66 | NW_020868548.1 | 338068 | T |
| A67 | NW_020868553.1 | 202039 | A |
| A68 | NW_020868557.1 | 44933 | G |
| A69 | NW_020868557.1 | 884071 | A |
| A70 | NW_020868561.1 | 60426 | T |
| A71 | NW_020868562.1 | 39367 | A |
| A72 | NW_020868566.1 | 70832 | G |
| A73 | NW_020868568.1 | 620385 | C |
| A74 | NW_020868571.1 | 327147 | C |
| A75 | NW_020868578.1 | 487353 | A |
| A76 | NW_020868579.1 | 99291 | A |
| A77 | NW_020868581.1 | 1175016 | T |
| A78 | NW_020868582.1 | 77505 | T |
| A79 | NW_020868588.1 | 53104 | G |
| A80 | NW_020868590.1 | 166732 | A |
| A81 | NW_020868593.1 | 219106 | T |
| A82 | NW_020868596.1 | 189463 | C |
| A83 | NW_020868596.1 | 400219 | C |
| A84 | NW_020868602.1 | 363304 | C |
| A85 | NW_020868605.1 | 384094 | C |
| A86 | NW_020868607.1 | 690423 | A |
| A87 | NW_020868608.1 | 315923 | A |
| A88 | NW_020868610.1 | 124096 | G |
| A89 | NW_020868611.1 | 658095 | C |
| A90 | NW_020868617.1 | 126431 | A |
| A91 | NW_020868624.1 | 544119 | T |
| A92 | NW_020868626.1 | 18002 | C |
| A93 | NW_020868628.1 | 455458 | C |
| A94 | NW_020868629.1 | 448174 | A |
| A95 | NW_020868631.1 | 381221 | G |
| A96 | NW_020868637.1 | 10141 | T |
| A97 | NW_020868637.1 | 826121 | C |
| A98 | NW_020868641.1 | 543888 | C |
| A99 | NW_020868646.1 | 984476 | A |
| A100 | NW_020868653.1 | 385080 | T |
| A101 | NW_020868658.1 | 157072 | C |
| A102 | NW_020868662.1 | 201191 | G |
| A103 | NW_020868666.1 | 3747 | C |
| A104 | NW_020868667.1 | 314864 | A |
| A105 | NW_020868668.1 | 170556 | T |
| A106 | NW_020868669.1 | 444922 | T |
| A107 | NW_020868671.1 | 56108 | C |
| A108 | NW_020868672.1 | 1050794 | G |
| A109 | NW_020868675.1 | 714980 | A |
| A110 | NW_020868678.1 | 474798 | T |
| A111 | NW_020868680.1 | 240054 | A |
| A112 | NW_020868681.1 | 93241 | C |
| A113 | NW_020868685.1 | 56549 | A |
| A114 | NW_020868686.1 | 375097 | T |
| A115 | NW_020868688.1 | 544675 | T |
| A116 | NW_020868695.1 | 15856 | A |
| A117 | NW_020868700.1 | 560346 | T |
| A118 | NW_020868703.1 | 28437 | T |
| A119 | NW_020868707.1 | 513384 | T |
| A120 | NW_020868714.1 | 369543 | C |
| A121 | NW_020868718.1 | 424008 | A |
| A122 | NW_020868733.1 | 733573 | C |
| A123 | NW_020868737.1 | 1236920 | T |
| A124 | NW_020868740.1 | 204092 | A |
| A125 | NW_020868745.1 | 274449 | G |
| A126 | NW_020868748.1 | 418551 | C |
| A127 | NW_020868752.1 | 697563 | A |
| A128 | NW_020868753.1 | 255840 | G |
| A129 | NW_020868760.1 | 67162 | C |
| A130 | NW_020868770.1 | 87546 | C |
| A131 | NW_020868772.1 | 349040 | A |
| A132 | NW_020868782.1 | 1104469 | A |
| A133 | NW_020868784.1 | 41074 | G |
| A134 | NW_020868786.1 | 235309 | T |
| A135 | NW_020868790.1 | 963672 | T |
| A136 | NW_020868792.1 | 706674 | G |
| A137 | NW_020868795.1 | 18926 | T |
| A138 | NW_020868801.1 | 525112 | C |
| A139 | NW_020868804.1 | 600802 | A |
| A140 | NW_020868808.1 | 45427 | C |
| A141 | NW_020868810.1 | 900504 | T |
| A142 | NW_020868812.1 | 100914 | A |
| A143 | NW_020868818.1 | 356954 | G |
| A144 | NW_020868823.1 | 284130 | A |
| A145 | NW_020868825.1 | 829040 | T |
| A146 | NW_020868830.1 | 797200 | T |
| A147 | NW_020868835.1 | 410664 | G |
| A148 | NW_020868837.1 | 447441 | C |
| A149 | NW_020868847.1 | 13463 | A |
| A150 | NW_020868854.1 | 183182 | A |
| A151 | NW_020868857.1 | 566613 | G |
| A152 | NW_020868865.1 | 87671 | C |
| A153 | NW_020868868.1 | 838369 | T |
| A154 | NW_020868871.1 | 753036 | G |
| A155 | NW_020868874.1 | 499739 | A |
| A156 | NW_020868880.1 | 646919 | C |
| A157 | NW_020868889.1 | 255114 | T |
| A158 | NW_020868893.1 | 161379 | T |
| A159 | NW_020868894.1 | 285175 | C |
| A160 | NW_020868899.1 | 115023 | C |
| A161 | NW_020868905.1 | 584705 | A |
| A162 | NW_020868908.1 | 132232 | T |
| A163 | NW_020868911.1 | 95173 | T |
| A164 | NW_020868912.1 | 131088 | G |
| A165 | NW_020868913.1 | 769402 | A |
| A166 | NW_020868918.1 | 822337 | C |
| A167 | NW_020868919.1 | 487867 | T |
| A168 | NW_020868924.1 | 852288 | C |
| A169 | NW_020868927.1 | 289578 | A |
| A170 | NW_020868928.1 | 114100 | A |
| A171 | NW_020868937.1 | 239783 | C |
| A172 | NW_020868938.1 | 61909 | A |
| A173 | NW_020868944.1 | 23018 | C |
| A174 | NW_020868948.1 | 93510 | G |
| A175 | NW_020868949.1 | 769795 | T |
| A176 | NW_020868952.1 | 436114 | T |
| A177 | NW_020868954.1 | 14469 | G |
| A178 | NW_020868957.1 | 274325 | G |
| A179 | NW_020868964.1 | 27835 | G |
| A180 | NW_020868965.1 | 244652 | A |
| A181 | NW_020868966.1 | 1244464 | G |
| A182 | NW_020868967.1 | 21206 | T |
| A183 | NW_020868968.1 | 213316 | A |
| A184 | NW_020868971.1 | 212365 | T |
| A185 | NW_020868972.1 | 444904 | T |
| A186 | NW_020868973.1 | 445882 | C |
| A187 | NW_020868980.1 | 137253 | A |
| A188 | NW_020868983.1 | 355322 | C |
| A189 | NW_020868989.1 | 242600 | T |
| A190 | NW_020868990.1 | 212634 | T |
| A191 | NW_020868995.1 | 663455 | T |
| A192 | NW_020869007.1 | 190560 | T |
| A193 | NW_020869008.1 | 79923 | T |
| A194 | NW_020869010.1 | 515813 | T |
| A195 | NW_020869012.1 | 238833 | G |
| A196 | NW_020869018.1 | 5436 | G |
| A197 | NW_020869026.1 | 236454 | C |
| A198 | NW_020869029.1 | 842453 | T |
| A199 | NW_020869030.1 | 77495 | A |
| A200 | NW_020869032.1 | 111693 | C |
| A201 | NW_020869035.1 | 186364 | G |
| A202 | NW_020869038.1 | 668187 | T |
| A203 | NW_020869040.1 | 90679 | T |
| A204 | NW_020869050.1 | 1027398 | G |
| A205 | NW_020869050.1 | 2515901 | A |
| A206 | NW_020869052.1 | 135902 | A |
| A207 | NW_020869053.1 | 95158 | T |
| A208 | NW_020869057.1 | 566442 | C |
| A209 | NW_020869059.1 | 150208 | A |
| A210 | NW_020869060.1 | 385541 | T |
| A211 | NW_020869061.1 | 16172 | A |
| A212 | NW_020869061.1 | 984347 | A |
| A213 | NW_020869062.1 | 12095 | T |
| A214 | NW_020869063.1 | 15746 | G |
| A215 | NW_020869065.1 | 151494 | T |
| A216 | NW_020869072.1 | 630097 | A |
| A217 | NW_020869079.1 | 85340 | G |
| A218 | NW_020869081.1 | 96947 | G |
| A219 | NW_020869083.1 | 285832 | G |
| A220 | NW_020869090.1 | 383714 | G |
| A221 | NW_020869091.1 | 120317 | A |
| A222 | NW_020869093.1 | 256906 | C |
| A223 | NW_020869099.1 | 356862 | T |
| A224 | NW_020869103.1 | 304717 | T |
| A225 | NW_020869108.1 | 503392 | T |
| A226 | NW_020869111.1 | 495551 | T |
| A227 | NW_020869117.1 | 68334 | T |
| A228 | NW_020869118.1 | 587902 | T |
| A229 | NW_020869118.1 | 1339586 | G |
| A230 | NW_020869122.1 | 295995 | A |
| A231 | NW_020869124.1 | 969660 | A |
| A232 | NW_020869126.1 | 626743 | A |
| A233 | NW_020869127.1 | 288407 | T |
| A234 | NW_020869128.1 | 920132 | G |
| A235 | NW_020869130.1 | 477325 | A |
| A236 | NW_020869133.1 | 331647 | T |
| A237 | NW_020869133.1 | 1968767 | T |
| A238 | NW_020869146.1 | 68586 | T |
| A239 | NW_020869147.1 | 1519380 | C |
| A240 | NW_020869148.1 | 1045739 | T |
| A241 | NW_020869150.1 | 197717 | T |
| A242 | NW_020869152.1 | 468833 | T |
| A243 | NW_020869154.1 | 315791 | A |
| A244 | NW_020869157.1 | 258113 | T |
| A245 | NW_020869159.1 | 74009 | A |
| A246 | NW_020869160.1 | 178528 | A |
| A247 | NW_020869163.1 | 267396 | A |
| A248 | NW_020869165.1 | 45823 | C |
| A249 | NW_020869172.1 | 401640 | A |
| A250 | NW_020869174.1 | 496874 | C |
| A251 | NW_020869178.1 | 291562 | G |
| A252 | NW_020869180.1 | 102132 | A |
| A253 | NW_020869182.1 | 57276 | A |
| A254 | NW_020869184.1 | 131898 | G |
| A255 | NW_020869186.1 | 279482 | A |
| A256 | NW_020869190.1 | 1248409 | T |
| A257 | NW_020869194.1 | 579276 | A |
| A258 | NW_020869195.1 | 538066 | T |
| A259 | NW_020869197.1 | 278226 | C |
| A260 | NW_020869202.1 | 167338 | T |
| A261 | NW_020869204.1 | 88350 | C |
| A262 | NW_020869206.1 | 467618 | C |
| A263 | NW_020869209.1 | 88840 | T |
| A264 | NW_020869211.1 | 504632 | T |
| A265 | NW_020869213.1 | 598646 | C |
| A266 | NW_020869215.1 | 191818 | G |
| A267 | NW_020869216.1 | 325218 | T |
| A268 | NW_020869222.1 | 203432 | A |
| A269 | NW_020869224.1 | 44463 | C |
| A270 | NW_020869225.1 | 194229 | T |
| A271 | NW_020869230.1 | 319801 | A |
| A272 | NW_020869235.1 | 619438 | A |
| A273 | NW_020869245.1 | 29844 | C |
| A274 | NW_020869247.1 | 178195 | G |
| A275 | NW_020869250.1 | 533141 | C |
| A276 | NW_020869252.1 | 346320 | A |
| A277 | NW_020869259.1 | 279373 | T |
| A278 | NW_020869262.1 | 682757 | A |
| A279 | NW_020869262.1 | 2095888 | A |
| A280 | NW_020869263.1 | 287465 | G |
| A281 | NW_020869265.1 | 18189 | C |
| A282 | NW_020869267.1 | 367142 | T |
| A283 | NW_020869273.1 | 234389 | T |
| A284 | NW_020869276.1 | 220360 | A |
| A285 | NW_020869278.1 | 295920 | T |
| A286 | NW_020869282.1 | 185275 | G |
| A287 | NW_020869283.1 | 440986 | A |
| A288 | NW_020869284.1 | 490196 | T |
| A289 | NW_020869285.1 | 157686 | G |
| A290 | NW_020869288.1 | 429412 | T |
| A291 | NW_020869289.1 | 274845 | G |
| A292 | NW_020869295.1 | 605291 | C |
| A293 | NW_020869298.1 | 143780 | T |
| A294 | NW_020869300.1 | 232544 | T |
| A295 | NW_020869304.1 | 140044 | G |
| A296 | NW_020869305.1 | 240558 | A |
| A297 | NW_020869308.1 | 4607 | T |
| A298 | NW_020869308.1 | 1506719 | T |
| A299 | NW_020869310.1 | 357756 | G |
| A300 | NW_020869311.1 | 73517 | T |
| A301 | NW_020869313.1 | 456152 | C |
| A302 | NW_020869318.1 | 483958 | A |
| A303 | NW_020869321.1 | 403266 | T |
| A304 | NW_020869323.1 | 337949 | C |
| A305 | NW_020869324.1 | 10197 | G |
| A306 | NW_020869325.1 | 283412 | A |
| A307 | NW_020869327.1 | 132353 | C |
| A308 | NW_020869329.1 | 61020 | C |
| A309 | NW_020869332.1 | 545888 | C |
| A310 | NW_020869342.1 | 381408 | T |
| A311 | NW_020869346.1 | 33513 | T |
| A312 | NW_020869353.1 | 323536 | A |
| A313 | NW_020869354.1 | 38988 | T |
| A314 | NW_020869355.1 | 479221 | T |
| A315 | NW_020869356.1 | 46448 | A |
| A316 | NW_020869371.1 | 682895 | A |
| A317 | NW_020869373.1 | 130688 | A |
| A318 | NW_020869383.1 | 180097 | A |
| A319 | NW_020869389.1 | 390875 | A |
| A320 | NW_020869391.1 | 761833 | C |
| A321 | NW_020869393.1 | 19476 | G |
| A322 | NW_020869396.1 | 13983 | T |
| A323 | NW_020869401.1 | 414457 | C |
| A324 | NW_020869405.1 | 147861 | A |
| A325 | NW_020869424.1 | 289967 | T |
| A326 | NW_020869427.1 | 305167 | T |
| A327 | NW_020869429.1 | 1313885 | T |
| A328 | NW_020869431.1 | 314917 | A |
| A329 | NW_020869433.1 | 1151492 | A |
| A330 | NW_020869440.1 | 573441 | G |
| A331 | NW_020869449.1 | 129956 | G |
| A332 | NW_020869451.1 | 107123 | C |
| A333 | NW_020869455.1 | 153372 | T |
| A334 | NW_020869458.1 | 1530425 | G |
| A335 | NW_020869459.1 | 241467 | C |
| A336 | NW_020869464.1 | 196068 | G |
| A337 | NW_020869476.1 | 67798 | A |
| A338 | NW_020869481.1 | 435324 | G |
| A339 | NW_020869482.1 | 450092 | G |
| A340 | NW_020869492.1 | 232251 | A |
| A341 | NW_020869494.1 | 187744 | T |
| A342 | NW_020869495.1 | 609983 | A |
| A343 | NW_020869497.1 | 754535 | A |
| A344 | NW_020869498.1 | 108520 | A |
| A345 | NW_020869500.1 | 547421 | G |
| A346 | NW_020869513.1 | 68173 | A |
| A347 | NW_020869515.1 | 114840 | C |
| A348 | NW_020869516.1 | 252984 | T |
| A349 | NW_020869520.1 | 109706 | T |
| A350 | NW_020869521.1 | 90930 | T |
| A351 | NW_020869523.1 | 128332 | A |
| A352 | NW_020869524.1 | 112844 | G |
| A353 | NW_020869525.1 | 105077 | A |
| A354 | NW_020869527.1 | 1622985 | G |
| A355 | NW_020869529.1 | 673491 | T |
| A356 | NW_020869531.1 | 398475 | C |
| A357 | NW_020869534.1 | 76100 | T |
| A358 | NW_020869537.1 | 156702 | T |
| A359 | NW_020869539.1 | 754062 | A |
| A360 | NW_020869540.1 | 457209 | A |
| A361 | NW_020869543.1 | 89233 | G |
| A362 | NW_020869544.1 | 231706 | A |
| A363 | NW_020869548.1 | 308392 | G |
| A364 | NW_020869550.1 | 104015 | A |
| A365 | NW_020869551.1 | 229330 | T |
| A366 | NW_020869553.1 | 213239 | T |
| A367 | NW_020869557.1 | 465347 | T |
| A368 | NW_020869560.1 | 155981 | T |
| A369 | NW_020869562.1 | 482588 | T |
| A370 | NW_020869563.1 | 63057 | T |
| A371 | NW_020869576.1 | 4657 | T |
| A372 | NW_020869578.1 | 755990 | A |
| A373 | NW_020869588.1 | 69872 | C |
| A374 | NW_020869589.1 | 357316 | G |
| A375 | NW_020869593.1 | 172080 | G |
| A376 | NW_020869594.1 | 79931 | T |
| A377 | NW_020869600.1 | 1131812 | A |
| A378 | NW_020869604.1 | 1160252 | C |
| A379 | NW_020869607.1 | 169135 | A |
| A380 | NW_020869611.1 | 441872 | T |
| A381 | NW_020869612.1 | 249411 | A |
| A382 | NW_020869615.1 | 671966 | T |
| A383 | NW_020869623.1 | 553625 | T |
| A384 | NW_020869625.1 | 643058 | C |
| A385 | NW_020869631.1 | 437006 | G |
| A386 | NW_020869633.1 | 439529 | A |
| A387 | NW_020869635.1 | 285866 | T |
| A388 | NW_020869641.1 | 246268 | A |
| A389 | NW_020869643.1 | 435356 | A |
| A390 | NW_020869644.1 | 261337 | A |
| A391 | NW_020869647.1 | 185967 | A |
| A392 | NW_020869648.1 | 83711 | C |
| A393 | NW_020869652.1 | 1254 | C |
| A394 | NW_020869654.1 | 56215 | T |
| A395 | NW_020869657.1 | 503413 | T |
| A396 | NW_020869660.1 | 995510 | G |
| A397 | NW_020869666.1 | 321912 | T |
| A398 | NW_020869667.1 | 644265 | A |
| A399 | NW_020869668.1 | 133427 | A |
| A400 | NW_020869670.1 | 204712 | T |
| A401 | NW_020869686.1 | 165669 | A |
| A402 | NW_020869688.1 | 625902 | T |
| A403 | NW_020869691.1 | 152555 | C |
| A404 | NW_020869696.1 | 630126 | T |
| A405 | NW_020869697.1 | 58723 | T |
| A406 | NW_020869700.1 | 66246 | T |
| A407 | NW_020869701.1 | 27637 | C |
| A408 | NW_020869702.1 | 29123 | C |
| A409 | NW_020869703.1 | 162375 | C |
| A410 | NW_020869704.1 | 448770 | A |
| A411 | NW_020869708.1 | 40076 | A |
| A412 | NW_020869712.1 | 138510 | T |
| A413 | NW_020869714.1 | 206107 | C |
| A414 | NW_020869716.1 | 92107 | C |
| A415 | NW_020869716.1 | 1012538 | T |
| A416 | NW_020869724.1 | 142019 | A |
| A417 | NW_020869733.1 | 193592 | G |
| A418 | NW_020869735.1 | 510602 | G |
| A419 | NW_020869738.1 | 464112 | T |
| A420 | NW_020869741.1 | 622403 | C |
| A421 | NW_020869744.1 | 308506 | A |
| A422 | NW_020869749.1 | 255697 | C |
| A423 | NW_020869750.1 | 1111542 | G |
| A424 | NW_020869755.1 | 55998 | C |
| A425 | NW_020869760.1 | 602305 | C |
| A426 | NW_020869768.1 | 239388 | C |
| A427 | NW_020869769.1 | 419897 | A |
| A428 | NW_020869770.1 | 196315 | T |
| A429 | NW_020869773.1 | 221972 | A |
| A430 | NW_020869783.1 | 740459 | T |
| A431 | NW_020869785.1 | 455483 | T |
| A432 | NW_020869786.1 | 658075 | T |
| A433 | NW_020869791.1 | 473579 | C |
| A434 | NW_020869797.1 | 395020 | A |
| A435 | NW_020869801.1 | 207821 | A |
| A436 | NW_020869804.1 | 608289 | A |
| A437 | NW_020869806.1 | 649130 | G |
| A438 | NW_020869810.1 | 301451 | G |
| A439 | NW_020869813.1 | 309392 | C |
| A440 | NW_020869817.1 | 537023 | C |
| A441 | NW_020869822.1 | 145802 | C |
| A442 | NW_020869822.1 | 938210 | A |
| A443 | NW_020869823.1 | 227842 | T |
| A444 | NW_020869824.1 | 47037 | G |
| A445 | NW_020869826.1 | 236398 | G |
| A446 | NW_020869829.1 | 120232 | T |
| A447 | NW_020869833.1 | 410023 | T |
| A448 | NW_020869838.1 | 235937 | G |
| A449 | NW_020869845.1 | 158645 | G |
| A450 | NW_020869847.1 | 478253 | A |
| A451 | NW_020869849.1 | 22818 | C |
| A452 | NW_020869849.1 | 275197 | T |
| A453 | NW_020869851.1 | 325471 | C |
| A454 | NW_020869852.1 | 485975 | G |
| A455 | NW_020869857.1 | 93415 | A |
| A456 | NW_020869858.1 | 32972 | A |
| A457 | NW_020869858.1 | 1495939 | C |
| A458 | NW_020869860.1 | 998351 | C |
| A459 | NW_020869861.1 | 108326 | A |
| A460 | NW_020869862.1 | 139247 | T |
| A461 | NW_020869873.1 | 135004 | G |
| A462 | NW_020869878.1 | 86614 | A |
| A463 | NW_020869881.1 | 374778 | T |
| A464 | NW_020869884.1 | 59388 | G |
| A465 | NW_020869885.1 | 271631 | A |
| A466 | NW_020869886.1 | 396151 | G |
| A467 | NW_020869892.1 | 693641 | T |
| A468 | NW_020869894.1 | 949226 | A |
| A469 | NW_020869898.1 | 198025 | T |
| A470 | NW_020869901.1 | 174834 | A |
| A471 | NW_020869903.1 | 605051 | T |
| A472 | NW_020869904.1 | 148553 | A |
| A473 | NW_020869906.1 | 332026 | A |
| A474 | NW_020869908.1 | 369526 | A |
| A475 | NW_020869914.1 | 196197 | T |
| A476 | NW_020869925.1 | 55689 | C |
| A477 | NW_020869926.1 | 193769 | A |
| A478 | NW_020869927.1 | 446694 | T |
| A479 | NW_020869928.1 | 198716 | T |
| A480 | NW_020869929.1 | 368640 | C |
| A481 | NW_020869933.1 | 66316 | T |
| A482 | NW_020869934.1 | 75398 | G |
| A483 | NW_020869935.1 | 210622 | T |
| A484 | NW_020869938.1 | 167689 | T |
| A485 | NW_020869940.1 | 419989 | G |
| A486 | NW_020869942.1 | 137259 | A |
| A487 | NW_020869943.1 | 58979 | A |
| A488 | NW_020869951.1 | 198725 | T |
| A489 | NW_020869955.1 | 518852 | T |
| A490 | NW_020869956.1 | 236663 | T |
| A491 | NW_020869959.1 | 415938 | C |
| A492 | NW_020869964.1 | 186302 | G |
| A493 | NW_020869965.1 | 191840 | T |
| A494 | NW_020869967.1 | 100313 | G |
| A495 | NW_020869970.1 | 186047 | G |
| A496 | NW_020869974.1 | 286513 | A |
| A497 | NW_020869977.1 | 219598 | A |
| A498 | NW_020869982.1 | 756860 | G |
| A499 | NW_020869989.1 | 42865 | A |
| A500 | NW_020869991.1 | 102867 | C |
| A501 | NW_020869992.1 | 236689 | A |
| A502 | NW_020869993.1 | 288192 | T |
| A503 | NW_020869997.1 | 634880 | T |
| A504 | NW_020870005.1 | 329796 | C |
| A505 | NW_020870006.1 | 146826 | A |
| A506 | NW_020870007.1 | 759373 | T |
| A507 | NW_020870013.1 | 95485 | G |
| A508 | NW_020870016.1 | 788722 | G |
| A509 | NW_020870018.1 | 134513 | T |
| A510 | NW_020870019.1 | 564956 | C |
| A511 | NW_020870020.1 | 201467 | G |
| A512 | NW_020870026.1 | 7096 | G |
| A513 | NW_020870026.1 | 1669018 | T |
| A514 | NW_020870026.1 | 2488115 | T |
| A515 | NW_020870028.1 | 978173 | T |
| A516 | NW_020870030.1 | 928403 | T |
| A517 | NW_020870032.1 | 106657 | G |
| A518 | NW_020870033.1 | 112027 | A |
| A519 | NW_020870041.1 | 171362 | G |
| A520 | NW_020870044.1 | 71914 | A |
| A521 | NW_020870048.1 | 142266 | T |
| A522 | NW_020870051.1 | 95835 | T |
| A523 | NW_020870053.1 | 259357 | T |
| A524 | NW_020870058.1 | 179073 | C |
| A525 | NW_020870059.1 | 12160 | A |
| A526 | NW_020870059.1 | 931033 | A |
| A527 | NW_020870061.1 | 548323 | T |
| A528 | NW_020870070.1 | 240228 | T |
| A529 | NW_020870074.1 | 148267 | A |
| A530 | NW_020870075.1 | 549794 | T |
| A531 | NW_020870077.1 | 314531 | G |
| A532 | NW_020870088.1 | 853841 | G |
| A533 | NW_020870091.1 | 1278610 | T |
| A534 | NW_020870092.1 | 412790 | G |
| A535 | NW_020870094.1 | 135391 | C |
| A536 | NW_020870094.1 | 949963 | A |
| A537 | NW_020870095.1 | 421338 | T |
| A538 | NW_020870097.1 | 19715 | T |
| A539 | NW_020870098.1 | 103052 | G |
| A540 | NW_020870100.1 | 904 | T |
| A541 | NW_020870101.1 | 141087 | A |
| A542 | NW_020870106.1 | 1086139 | A |
| A543 | NW_020870107.1 | 77193 | G |
| A544 | NW_020870108.1 | 249667 | A |
| A545 | NW_020870110.1 | 65015 | G |
| A546 | NW_020870116.1 | 490209 | G |
| A547 | NW_020870118.1 | 105026 | A |
| A548 | NW_020870125.1 | 1187034 | T |
| A549 | NW_020870128.1 | 914073 | A |
| A550 | NW_020870129.1 | 381078 | C |
| A551 | NW_020870130.1 | 588755 | G |
| A552 | NW_020870134.1 | 15209 | T |
| A553 | NW_020870136.1 | 28570 | C |
| A554 | NW_020870139.1 | 421228 | A |
| A555 | NW_020870146.1 | 14190 | C |
| A556 | NW_020870146.1 | 640630 | T |
| A557 | NW_020870153.1 | 491192 | A |
| A558 | NW_020870155.1 | 601687 | A |
| A559 | NW_020870157.1 | 41102 | T |
| A560 | NW_020870163.1 | 154714 | G |
| A561 | NW_020870168.1 | 184593 | C |
| A562 | NW_020870169.1 | 210426 | C |
| A563 | NW_020870170.1 | 48911 | G |
| A564 | NW_020870173.1 | 504375 | T |
| A565 | NW_020870179.1 | 15615 | T |
| A566 | NW_020870180.1 | 187409 | A |
| A567 | NW_020870182.1 | 514060 | A |
| A568 | NW_020870183.1 | 448848 | C |
| A569 | NW_020870185.1 | 931364 | C |
| A570 | NW_020870185.1 | 1056348 | G |
| A571 | NW_020870195.1 | 139985 | A |
| A572 | NW_020870202.1 | 609688 | C |
| A573 | NW_020870204.1 | 748743 | T |
| A574 | NW_020870205.1 | 98140 | A |
| A575 | NW_020870207.1 | 281234 | T |
| A576 | NW_020870212.1 | 37595 | T |
| A577 | NW_020870216.1 | 450201 | T |
| A578 | NW_020870217.1 | 52666 | C |
| A579 | NW_020870219.1 | 1200363 | T |
| A580 | NW_020870220.1 | 36279 | G |
| A581 | NW_020870222.1 | 976622 | C |
| A582 | NW_020870226.1 | 261017 | T |
| A583 | NW_020870227.1 | 366896 | G |
| A584 | NW_020870230.1 | 143445 | G |
| A585 | NW_020870233.1 | 140603 | G |
| A586 | NW_020870239.1 | 428948 | A |
| A587 | NW_020870240.1 | 142188 | A |
| A588 | NW_020870241.1 | 902788 | A |
| A589 | NW_020870243.1 | 24603 | T |
| A590 | NW_020870248.1 | 834331 | C |
| A591 | NW_020870271.1 | 532344 | C |
| A592 | NW_020870272.1 | 450477 | G |
| A593 | NW_020870274.1 | 570408 | C |
| A594 | NW_020870276.1 | 441676 | T |
| A595 | NW_020870279.1 | 174080 | A |
| A596 | NW_020870287.1 | 245420 | T |
| A597 | NW_020870292.1 | 32364 | G |
| A598 | NW_020870294.1 | 214578 | T |
| A599 | NW_020870295.1 | 853487 | C |
| A600 | NW_020870296.1 | 442443 | T |
| A601 | NW_020870297.1 | 1570349 | T |
| A602 | NW_020870301.1 | 958295 | C |
| A603 | NW_020870307.1 | 781840 | C |
| A604 | NW_020870308.1 | 314357 | T |
| A605 | NW_020870309.1 | 15767 | T |
| A606 | NW_020870312.1 | 167923 | G |
| A607 | NW_020870314.1 | 124993 | C |
| A608 | NW_020870314.1 | 758904 | G |
| A609 | NW_020870315.1 | 300225 | A |
| A610 | NW_020870315.1 | 1853475 | G |
| A611 | NW_020870319.1 | 82623 | A |
| A612 | NW_020870322.1 | 796636 | G |
| A613 | NW_020870324.1 | 114373 | T |
| A614 | NW_020870325.1 | 236786 | A |
| A615 | NW_020870328.1 | 276258 | T |
| A616 | NW_020870336.1 | 42391 | A |
| A617 | NW_020870338.1 | 86368 | A |
| A618 | NW_020870339.1 | 1083869 | T |
| A619 | NW_020870340.1 | 423934 | T |
| A620 | NW_020870344.1 | 440439 | C |
| A621 | NW_020870345.1 | 53727 | T |
| A622 | NW_020870350.1 | 77221 | T |
| A623 | NW_020870359.1 | 608808 | T |
| A624 | NW_020870362.1 | 211840 | G |
| A625 | NW_020870363.1 | 30185 | T |
| A626 | NW_020870365.1 | 441006 | G |
| A627 | NW_020870368.1 | 1042810 | T |
| A628 | NW_020870373.1 | 263147 | T |
| A629 | NW_020870376.1 | 412874 | T |
| A630 | NW_020870378.1 | 587168 | A |
| A631 | NW_020870379.1 | 511871 | C |
| A632 | NW_020870383.1 | 403961 | G |
| A633 | NW_020870389.1 | 613984 | C |
| A634 | NW_020870392.1 | 175642 | T |
| A635 | NW_020870401.1 | 334819 | T |
| A636 | NW_020870403.1 | 768147 | C |
| A637 | NW_020870406.1 | 84776 | G |
| A638 | NW_020870418.1 | 502089 | T |
| A639 | NW_020870419.1 | 514601 | G |
| A640 | NW_020870426.1 | 848406 | T |
| A641 | NW_020870434.1 | 97027 | T |
| A642 | NW_020870436.1 | 529127 | A |
| A643 | NW_020870437.1 | 661746 | G |
| A644 | NW_020870439.1 | 604056 | G |
| A645 | NW_020870445.1 | 110461 | A |
| A646 | NW_020870449.1 | 788764 | G |
| A647 | NW_020870451.1 | 529328 | A |
| A648 | NW_020870456.1 | 207327 | A |
| A649 | NW_020870462.1 | 71524 | T |
| A650 | NW_020870471.1 | 41622 | T |
| A651 | NW_020870472.1 | 1194008 | G |
| A652 | NW_020870475.1 | 896998 | A |
| A653 | NW_020870482.1 | 203153 | C |
| A654 | NW_020870489.1 | 50576 | G |
| A655 | NW_020870491.1 | 787670 | G |
| A656 | NW_020870492.1 | 628522 | A |
| A657 | NW_020870500.1 | 378380 | G |
| A658 | NW_020870506.1 | 622108 | A |
| A659 | NW_020870509.1 | 158927 | A |
| A660 | NW_020870511.1 | 326844 | A |
| A661 | NW_020870514.1 | 454794 | G |
| A662 | NW_020870523.1 | 109234 | G |
| A663 | NW_020870526.1 | 273784 | G |
| A664 | NW_020870528.1 | 978295 | G |
| A665 | NW_020870529.1 | 1156367 | A |
| A666 | NW_020870531.1 | 1587095 | C |
| A667 | NW_020870536.1 | 173463 | C |
| A668 | NW_020870544.1 | 530509 | T |
| A669 | NW_020870547.1 | 216863 | G |
| A670 | NW_020870553.1 | 189389 | A |
| A671 | NW_020870555.1 | 460066 | G |
| A672 | NW_020870557.1 | 482653 | A |
| A673 | NW_020870561.1 | 8939 | G |
| A674 | NW_020870563.1 | 139314 | G |
| A675 | NW_020870569.1 | 160018 | G |
| A676 | NW_020870572.1 | 412365 | T |
| A677 | NW_020870577.1 | 106097 | T |
| A678 | NW_020870579.1 | 210420 | C |
| A679 | NW_020870588.1 | 190150 | A |
| A680 | NW_020870595.1 | 157158 | A |
| A681 | NW_020870599.1 | 526289 | A |
| A682 | NW_020870603.1 | 249534 | G |
| A683 | NW_020870606.1 | 466247 | T |
| A684 | NW_020870607.1 | 447579 | A |
| A685 | NW_020870609.1 | 153516 | T |
| A686 | NW_020870613.1 | 311909 | T |
| A687 | NW_020870614.1 | 371835 | T |
| A688 | NW_020870615.1 | 171357 | A |
| A689 | NW_020870617.1 | 213457 | T |
| A690 | NW_020870617.1 | 450928 | C |
| A691 | NW_020870621.1 | 17734 | C |
| A692 | NW_020870624.1 | 24937 | T |
| A693 | NW_020870627.1 | 31374 | T |
| A694 | NW_020870630.1 | 812864 | A |
| A695 | NW_020870631.1 | 377778 | A |
| A696 | NW_020870638.1 | 231843 | G |
| A697 | NW_020870643.1 | 140915 | T |
| A698 | NW_020870644.1 | 168572 | G |
| A699 | NW_020870645.1 | 886654 | T |
| A700 | NW_020870646.1 | 300178 | A |
| A701 | NW_020870651.1 | 849718 | A |
| A702 | NW_020870656.1 | 64157 | G |
| A703 | NW_020870657.1 | 476305 | C |
| A704 | NW_020870658.1 | 679268 | G |
| A705 | NW_020870669.1 | 324779 | C |
| A706 | NW_020870673.1 | 522722 | A |
| A707 | NW_020870675.1 | 98201 | A |
| A708 | NW_020870679.1 | 195851 | A |
| A709 | NW_020870681.1 | 210076 | G |
| A710 | NW_020870685.1 | 327314 | A |
| A711 | NW_020870688.1 | 601578 | T |
| A712 | NW_020870689.1 | 792452 | T |
| A713 | NW_020870699.1 | 1258808 | T |
| A714 | NW_020870703.1 | 261441 | A |
| A715 | NW_020870704.1 | 208885 | C |
| A716 | NW_020870707.1 | 623747 | G |
| A717 | NW_020870710.1 | 24937 | A |
| A718 | NW_020870711.1 | 779232 | G |
| A719 | NW_020870724.1 | 36898 | A |
| A720 | NW_020870725.1 | 474640 | T |
| A721 | NW_020870734.1 | 1438006 | T |
| A722 | NW_020870736.1 | 282833 | A |
| A723 | NW_020870737.1 | 340464 | G |
| A724 | NW_020870746.1 | 127976 | C |
| A725 | NW_020870747.1 | 644055 | T |
| A726 | NW_020870750.1 | 123378 | A |
| A727 | NW_020870754.1 | 43620 | G |
| A728 | NW_020870755.1 | 48364 | T |
| A729 | NW_020870758.1 | 134255 | A |
| A730 | NW_020870760.1 | 98156 | C |
| A731 | NW_020870761.1 | 80236 | T |
| A732 | NW_020870761.1 | 731915 | A |
| A733 | NW_020870762.1 | 42894 | C |
| A734 | NW_020870763.1 | 196953 | T |
| A735 | NW_020870764.1 | 371038 | A |
| A736 | NW_020870771.1 | 263880 | G |
| A737 | NW_020870777.1 | 904225 | C |
| A738 | NW_020870779.1 | 121363 | A |
| A739 | NW_020870781.1 | 1006246 | A |
| A740 | NW_020870786.1 | 549851 | C |
| A741 | NW_020870788.1 | 138833 | C |
| A742 | NW_020870789.1 | 424358 | C |
| A743 | NW_020870796.1 | 190994 | G |
| A744 | NW_020870799.1 | 117724 | A |
| A745 | NW_020870800.1 | 615584 | C |
| A746 | NW_020870801.1 | 81823 | A |
| A747 | NW_020870805.1 | 127710 | T |
| A748 | NW_020870815.1 | 60551 | T |
| A749 | NW_020870816.1 | 86982 | C |
| A750 | NW_020870819.1 | 143332 | C |
| A751 | NW_020870820.1 | 394751 | T |
| A752 | NW_020870822.1 | 45912 | C |
| A753 | NW_020870823.1 | 498487 | A |
| A754 | NW_020870824.1 | 454950 | G |
| A755 | NW_020870830.1 | 93784 | T |
| A756 | NW_020870833.1 | 49888 | A |
| A757 | NW_020870834.1 | 142004 | G |
| A758 | NW_020870844.1 | 496843 | C |
| A759 | NW_020870846.1 | 252894 | T |
| A760 | NW_020870848.1 | 316954 | G |
| A761 | NW_020870849.1 | 365864 | T |
| A762 | NW_020870855.1 | 669693 | C |
| A763 | NW_020870859.1 | 736521 | A |
| A764 | NW_020870866.1 | 539344 | A |
| A765 | NW_020870873.1 | 93755 | C |
| A766 | NW_020870881.1 | 403210 | A |
| A767 | NW_020870886.1 | 61479 | T |
| A768 | NW_020870888.1 | 126022 | A |
| A769 | NW_020870896.1 | 843071 | A |
| A770 | NW_020870899.1 | 371515 | A |
| A771 | NW_020870901.1 | 726880 | A |
| A772 | NW_020870904.1 | 82149 | A |
| A773 | NW_020870905.1 | 8359 | A |
| A774 | NW_020870906.1 | 34374 | G |
| A775 | NW_020870907.1 | 160250 | C |
| A776 | NW_020870910.1 | 114269 | T |
| A777 | NW_020870911.1 | 1202067 | A |
| A778 | NW_020870914.1 | 226445 | C |
| A779 | NW_020870915.1 | 119073 | T |
| A780 | NW_020870916.1 | 201416 | A |
| A781 | NW_020870925.1 | 128551 | T |
| A782 | NW_020870928.1 | 1256299 | C |
| A783 | NW_020870929.1 | 344018 | C |
| A784 | NW_020870930.1 | 32068 | T |
| A785 | NW_020870937.1 | 201808 | A |
| A786 | NW_020870943.1 | 259344 | A |
| A787 | NW_020870947.1 | 381113 | C |
| A788 | NW_020870951.1 | 142935 | A |
| A789 | NW_020870952.1 | 268162 | A |
| A790 | NW_020870957.1 | 200803 | A |
| A791 | NW_020870969.1 | 423004 | A |
| A792 | NW_020870972.1 | 348449 | A |
| A793 | NW_020870977.1 | 7134 | T |
| A794 | NW_020870981.1 | 698781 | A |
| A795 | NW_020870983.1 | 149840 | T |
| A796 | NW_020870985.1 | 101089 | A |
| A797 | NW_020870988.1 | 1028385 | G |
| A798 | NW_020870989.1 | 372077 | C |
| A799 | NW_020870992.1 | 669198 | C |
| A800 | NW_020870999.1 | 840136 | A |
| A801 | NW_020871003.1 | 12395 | T |
| A802 | NW_020871005.1 | 352834 | T |
| A803 | NW_020871007.1 | 536053 | A |
| A804 | NW_020871008.1 | 406943 | A |
| A805 | NW_020871010.1 | 33070 | C |
| A806 | NW_020871014.1 | 341183 | T |
| A807 | NW_020871018.1 | 68100 | A |
| A808 | NW_020871022.1 | 161633 | A |
| A809 | NW_020871022.1 | 920136 | C |
| A810 | NW_020871023.1 | 232362 | C |
| A811 | NW_020871040.1 | 42967 | T |
| A812 | NW_020871040.1 | 1623245 | A |
| A813 | NW_020871044.1 | 198922 | C |
| A814 | NW_020871052.1 | 3275 | C |
| A815 | NW_020871056.1 | 184085 | T |
| A816 | NW_020871062.1 | 442063 | C |
| A817 | NW_020871071.1 | 60371 | C |
| A818 | NW_020871072.1 | 861708 | A |
| A819 | NW_020871078.1 | 489262 | A |
| A820 | NW_020871079.1 | 238412 | A |
| A821 | NW_020871081.1 | 136181 | T |
| A822 | NW_020871088.1 | 243252 | C |
| A823 | NW_020871090.1 | 590205 | T |
| A824 | NW_020871091.1 | 125020 | C |
| A825 | NW_020871094.1 | 1286443 | C |
| A826 | NW_020871095.1 | 32971 | G |
| A827 | NW_020871098.1 | 25541 | T |
| A828 | NW_020871100.1 | 135446 | T |
| A829 | NW_020871103.1 | 161383 | A |
| A830 | NW_020871109.1 | 20641 | T |
| A831 | NW_020871117.1 | 159404 | C |
| A832 | NW_020871125.1 | 179278 | G |
| A833 | NW_020871126.1 | 407792 | C |
| A834 | NW_020871133.1 | 22239 | T |
| A835 | NW_020871134.1 | 53044 | G |
| A836 | NW_020871135.1 | 194737 | A |
| A837 | NW_020871141.1 | 214649 | T |
| A838 | NW_020871164.1 | 513635 | A |
| A839 | NW_020871165.1 | 47635 | G |
| A840 | NW_020871170.1 | 164077 | G |
| A841 | NW_020871172.1 | 29669 | G |
| A842 | NW_020871172.1 | 280194 | T |
| A843 | NW_020871193.1 | 301731 | A |
| A844 | NW_020871204.1 | 18898 | G |
| A845 | NW_020871204.1 | 699554 | G |
| A846 | NW_020871214.1 | 56265 | A |
| A847 | NW_020871215.1 | 167934 | A |
| A848 | NW_020871223.1 | 89867 | T |
| A849 | NW_020871226.1 | 184460 | G |
| A850 | NW_020871232.1 | 812344 | T |
| A851 | NW_020871246.1 | 86514 | T |
| A852 | NW_020871249.1 | 645727 | T |
| A853 | NW_020871259.1 | 31134 | C |
| A854 | NW_020871259.1 | 548965 | T |
| A855 | NW_020871267.1 | 160387 | A |
| A856 | NW_020871270.1 | 15014 | A |
| A857 | NW_020871279.1 | 410484 | T |
| A858 | NW_020871293.1 | 73540 | T |
| A859 | NW_020871323.1 | 261651 | T |
| A860 | NW_020871329.1 | 32950 | A |
| A861 | NW_020871336.1 | 84187 | C |
| A862 | NW_020871339.1 | 129572 | A |
| A863 | NW_020871371.1 | 268164 | C |
| A864 | NW_020871391.1 | 321316 | G |
| A865 | NW_020871414.1 | 213183 | A |
| A866 | NW_020871420.1 | 70165 | A |
| A867 | NW_020871423.1 | 207593 | T |
| A868 | NW_020871428.1 | 607090 | C |
| A869 | NW_020871431.1 | 111207 | A |
| A870 | NW_020871451.1 | 48937 | G |
| A871 | NW_020871455.1 | 5884 | G |
| A872 | NW_020871463.1 | 131634 | A |
| A873 | NW_020871466.1 | 118754 | C |
| A874 | NW_020871470.1 | 1210883 | T |
| A875 | NW_020871485.1 | 235138 | C |
| A876 | NW_020871493.1 | 7663 | C |
| A877 | NW_020871497.1 | 62266 | A |
| A878 | NW_020871504.1 | 167741 | A |
| A879 | NW_020871507.1 | 103632 | A |
| A880 | NW_020871546.1 | 616652 | G |
| A881 | NW_020871553.1 | 283260 | A |
| A882 | NW_020871567.1 | 951158 | C |
| A883 | NW_020871575.1 | 132550 | A |
| A884 | NW_020871579.1 | 277779 | C |
| A885 | NW_020871588.1 | 1410174 | A |
| A886 | NW_020871610.1 | 445228 | C |
| A887 | NW_020871618.1 | 65888 | C |
| A888 | NW_020871622.1 | 208046 | G |
| A889 | NW_020871624.1 | 338162 | G |
| A890 | NW_020871628.1 | 24099 | A |
| A891 | NW_020871636.1 | 789835 | T |
| A892 | NW_020871663.1 | 864899 | C |
| A893 | NW_020871694.1 | 157672 | A |
| A894 | NW_020871694.1 | 598562 | T |
| A895 | NW_020871708.1 | 315056 | A |
| A896 | NW_020871718.1 | 17932 | C |
| A897 | NW_020871724.1 | 548921 | G |
| A898 | NW_020871725.1 | 39404 | A |
| A899 | NW_020871728.1 | 11563 | C |
| A900 | NW_020871737.1 | 299780 | T |
| A901 | NW_020871744.1 | 231757 | G |
| A902 | NW_020871810.1 | 222347 | A |
| A903 | NW_020871821.1 | 125920 | A |
| A904 | NW_020871827.1 | 210865 | A |
| A905 | NW_020871828.1 | 71490 | A |
| A906 | NW_020871828.1 | 206545 | T |
| A907 | NW_020871838.1 | 86248 | G |
| A908 | NW_020871841.1 | 714139 | C |
| A909 | NW_020871866.1 | 83780 | A |
| A910 | NW_020871871.1 | 54121 | G |
| A911 | NW_020871916.1 | 128659 | C |
| A912 | NW_020871918.1 | 96448 | C |
| A913 | NW_020871922.1 | 224337 | A |
| A914 | NW_020871923.1 | 517756 | C |
| A915 | NW_020871926.1 | 594840 | A |
| A916 | NW_020871936.1 | 52614 | G |
| A917 | NW_020871947.1 | 1458 | C |
| A918 | NW_020871947.1 | 802061 | G |
| A919 | NW_020871997.1 | 398814 | G |
| A920 | NW_020871998.1 | 174553 | A |
| A921 | NW_020871999.1 | 162163 | G |
| A922 | NW_020872010.1 | 19152 | A |
| A923 | NW_020872016.1 | 8103 | A |
| A924 | NW_020872066.1 | 302570 | A |
| A925 | NW_020872068.1 | 296227 | A |
| A926 | NW_020872073.1 | 181498 | T |
| A927 | NW_020872074.1 | 331440 | T |
| A928 | NW_020872085.1 | 233836 | G |
| A929 | NW_020872098.1 | 318997 | A |
| A930 | NW_020872101.1 | 164968 | A |
| A931 | NW_020872103.1 | 17541 | T |
| A932 | NW_020872111.1 | 168132 | C |
| A933 | NW_020872140.1 | 727925 | C |
| A934 | NW_020872143.1 | 501370 | C |
| A935 | NW_020872149.1 | 52221 | G |
| A936 | NW_020872174.1 | 16383 | C |
| A937 | NW_020872191.1 | 495543 | A |
| A938 | NW_020872203.1 | 281305 | A |
| A939 | NW_020872231.1 | 2844 | T |
| A940 | NW_020872232.1 | 86836 | C |
| A941 | NW_020872236.1 | 20858 | A |
| A942 | NW_020872248.1 | 62651 | T |
| A943 | NW_020872253.1 | 265725 | T |
| A944 | NW_020872260.1 | 630806 | C |
| A945 | NW_020872284.1 | 959702 | G |
| A946 | NW_020872300.1 | 198217 | G |
| A947 | NW_020872305.1 | 9972 | A |
| A948 | NW_020872307.1 | 126478 | A |
| A949 | NW_020872317.1 | 460853 | G |
| A950 | NW_020872325.1 | 451258 | G |
| A951 | NW_020872328.1 | 134269 | G |
| A952 | NW_020872336.1 | 73961 | T |
| A953 | NW_020872340.1 | 10631 | G |
| A954 | NW_020872366.1 | 310484 | A |
| A955 | NW_020872381.1 | 42586 | C |
| A956 | NW_020872385.1 | 479620 | C |
| A957 | NW_020872386.1 | 48885 | A |
| A958 | NW_020872393.1 | 61516 | A |
| A959 | NW_020872398.1 | 104539 | C |
| A960 | NW_020872401.1 | 95183 | C |
| A961 | NW_020872404.1 | 320548 | T |
| A962 | NW_020872407.1 | 103771 | C |
| A963 | NW_020872417.1 | 182317 | T |
| A964 | NW_020872420.1 | 1312303 | A |
| A965 | NW_020872422.1 | 186883 | C |
| A966 | NW_020872423.1 | 330780 | A |
| A967 | NW_020872424.1 | 213784 | T |
| A968 | NW_020872428.1 | 351239 | A |
| A969 | NW_020872431.1 | 357026 | T |
| A970 | NW_020872432.1 | 1097678 | G |
| A971 | NW_020872433.1 | 746864 | C |
| A972 | NW_020872437.1 | 562823 | C |
| A973 | NW_020872443.1 | 606210 | C |
| A974 | NW_020872445.1 | 199229 | T |
| A975 | NW_020872449.1 | 887334 | G |
| A976 | NW_020872450.1 | 639457 | A |
| A977 | NW_020872452.1 | 17117 | G |
| A978 | NW_020872454.1 | 508332 | T |
| A979 | NW_020872455.1 | 200462 | T |
| A980 | NW_020872463.1 | 26897 | C |
| A981 | NW_020872468.1 | 241027 | C |
| A982 | NW_020872475.1 | 181198 | A |
| A983 | NW_020872479.1 | 301337 | C |
| A984 | NW_020872482.1 | 209916 | G |
| A985 | NW_020872484.1 | 134422 | G |
| A986 | NW_020872485.1 | 551973 | C |
| A987 | NW_020872489.1 | 642409 | G |
| A988 | NW_020872494.1 | 330157 | A |
| A989 | NW_020872495.1 | 150710 | G |
| A990 | NW_020872504.1 | 341724 | T |
| A991 | NW_020872508.1 | 86300 | T |
| A992 | NW_020872512.1 | 46095 | T |
| A993 | NW_020872514.1 | 179357 | G |
| A994 | NW_020872521.1 | 388857 | T |
| A995 | NW_020872537.1 | 122521 | T |
| A996 | NW_020872540.1 | 44079 | T |
| A997 | NW_020872541.1 | 594195 | A |
| A998 | NW_020872545.1 | 125318 | G |
| A999 | NW_020872547.1 | 441451 | C |
| A1000 | NW_020872554.1 | 120989 | A |
| A1001 | NW_020872556.1 | 133330 | A |
| A1002 | NW_020872557.1 | 59929 | T |
| A1003 | NW_020872559.1 | 647842 | T |
| A1004 | NW_020872561.1 | 1094981 | C |
| A1005 | NW_020872567.1 | 278023 | C |
| A1006 | NW_020872570.1 | 176644 | C |
| A1007 | NW_020872571.1 | 41392 | A |
| A1008 | NW_020872573.1 | 513152 | C |
| A1009 | NW_020872576.1 | 65500 | G |
| A1010 | NW_020872577.1 | 1558 | G |
| A1011 | NW_020872597.1 | 104829 | C |
| A1012 | NW_020872599.1 | 529727 | G |
| A1013 | NW_020872601.1 | 199574 | A |
| A1014 | NW_020872607.1 | 564921 | T |
| A1015 | NW_020872608.1 | 845613 | A |
| A1016 | NW_020872610.1 | 597897 | A |
| A1017 | NW_020872620.1 | 39757 | T |
| A1018 | NW_020872622.1 | 990808 | A |
| A1019 | NW_020872632.1 | 402945 | T |
| A1020 | NW_020872634.1 | 380697 | A |
| A1021 | NW_020872640.1 | 166505 | A |
| A1022 | NW_020872642.1 | 587186 | A |
| A1023 | NW_020872655.1 | 169299 | A |
| A1024 | NW_020872656.1 | 8572 | G |
| A1025 | NW_020872667.1 | 148934 | G |
| A1026 | NW_020872670.1 | 185804 | C |
| A1027 | NW_020872671.1 | 101534 | G |
| A1028 | NW_020872676.1 | 9790 | T |
| A1029 | NW_020872677.1 | 127841 | C |
| A1030 | NW_020872686.1 | 459876 | C |
| A1031 | NW_020872687.1 | 58139 | A |
| A1032 | NW_020872693.1 | 420144 | G |
| A1033 | NW_020872696.1 | 961694 | G |
| A1034 | NW_020872697.1 | 109321 | A |
| A1035 | NW_020872701.1 | 158099 | A |
| A1036 | NW_020872701.1 | 1489216 | A |
| A1037 | NW_020872704.1 | 155165 | C |
| A1038 | NW_020872706.1 | 177621 | C |
| A1039 | NW_020872708.1 | 281903 | G |
| A1040 | NW_020872710.1 | 113125 | A |
| A1041 | NW_020872716.1 | 367373 | T |
| A1042 | NW_020872717.1 | 414534 | T |
| A1043 | NW_020872719.1 | 1271875 | A |
| A1044 | NW_020872720.1 | 190774 | A |
| A1045 | NW_020872722.1 | 361970 | C |
| A1046 | NW_020872723.1 | 195236 | A |
| A1047 | NW_020872726.1 | 836365 | C |
| A1048 | NW_020872728.1 | 303057 | T |
| A1049 | NW_020872734.1 | 968024 | A |
| A1050 | NW_020872735.1 | 351612 | C |
| A1051 | NW_020872737.1 | 2198 | C |
| A1052 | NW_020872741.1 | 423351 | G |
| A1053 | NW_020872742.1 | 460233 | A |
| A1054 | NW_020872743.1 | 57166 | T |
| A1055 | NW_020872745.1 | 33851 | G |
| A1056 | NW_020872748.1 | 94919 | T |
| A1057 | NW_020872749.1 | 342867 | T |
| A1058 | NW_020872755.1 | 237326 | A |
| A1059 | NW_020872761.1 | 399245 | C |
| A1060 | NW_020872762.1 | 737201 | C |
| A1061 | NW_020872766.1 | 1445301 | T |
| A1062 | NW_020872772.1 | 716456 | G |
| A1063 | NW_020872787.1 | 321840 | A |
| A1064 | NW_020872788.1 | 1175690 | A |
| A1065 | NW_020872800.1 | 31240 | C |
| A1066 | NW_020872804.1 | 433499 | T |
| A1067 | NW_020872806.1 | 62981 | A |
| A1068 | NW_020872808.1 | 631383 | A |
| A1069 | NW_020872814.1 | 175647 | C |
| A1070 | NW_020872817.1 | 167185 | T |
| A1071 | NW_020872820.1 | 21802 | A |
| A1072 | NW_020872825.1 | 298620 | A |
| A1073 | NW_020872826.1 | 162193 | G |
| A1074 | NW_020872833.1 | 83602 | C |
| A1075 | NW_020872837.1 | 7777 | T |
| A1076 | NW_020872850.1 | 464375 | C |
| A1077 | NW_020872851.1 | 587485 | G |
| A1078 | NW_020872855.1 | 124988 | G |
| A1079 | NW_020872859.1 | 315169 | A |
| A1080 | NW_020872860.1 | 64784 | T |
| A1081 | NW_020872872.1 | 21216 | C |
| A1082 | NW_020872873.1 | 460793 | C |
| A1083 | NW_020872875.1 | 292067 | A |
| A1084 | NW_020872876.1 | 242453 | C |
| A1085 | NW_020872878.1 | 132949 | A |
| A1086 | NW_020872879.1 | 232082 | T |
| A1087 | NW_020872882.1 | 709105 | A |
| A1088 | NW_020872894.1 | 488897 | C |
| A1089 | NW_020872896.1 | 2277 | A |
| A1090 | NW_020872906.1 | 3701 | C |
| A1091 | NW_020872907.1 | 1217777 | A |
| A1092 | NW_020872908.1 | 28747 | A |
| A1093 | NW_020872909.1 | 19217 | A |
| A1094 | NW_020872911.1 | 710111 | G |
| A1095 | NW_020872915.1 | 1377934 | A |
| A1096 | NW_020872922.1 | 259906 | T |
| A1097 | NW_020872926.1 | 3062 | G |
| A1098 | NW_020872930.1 | 934371 | G |
| A1099 | NW_020872942.1 | 180619 | T |
| A1100 | NW_020872943.1 | 301600 | G |
| A1101 | NW_020872943.1 | 571615 | A |
| A1102 | NW_020872945.1 | 27348 | C |
| A1103 | NW_020872948.1 | 56686 | C |
| A1104 | NW_020872955.1 | 320871 | G |
| A1105 | NW_020872956.1 | 643111 | A |
| A1106 | NW_020872958.1 | 627495 | T |
| A1107 | NW_020872964.1 | 62895 | A |
| A1108 | NW_020872966.1 | 504876 | A |
| A1109 | NW_020868585.1 | 48666 | G |
| A1110 | NW_020868711.1 | 1597 | C |
| A1111 | NW_020868724.1 | 18739 | A |
| A1112 | NW_020870268.1 | 30442 | T |
| A1113 | NW_020870909.1 | 36878 | T |
| A1114 | NW_020871119.1 | 4978 | T |
| A1115 | NW_020871190.1 | 9219 | A |
| A1116 | NW_020871592.1 | 55702 | A |
| A1117 | NW_020871677.1 | 5985 | T |
| A1118 | NW_020871704.1 | 4034 | G |
| A1119 | NW_020871851.1 | 665 | A |
| A1120 | NW_020872186.1 | 24415 | C |
| A1121 | NW_020872220.1 | 455 | A |
| A1122 | NW_020872295.1 | 19691 | A |
| A1123 | NW_020872351.1 | 68606 | A |
| A1124 | NW_020872793.1 | 70352 | G |
| A1125 | NW_020872803.1 | 35946 | T |

2. A molecular probe for use in Litopenaeus vannamei family mixed breeding trait evaluation, wherein the molecular probe is a single-stranded DNA probe comprising 1 to 4 distinct nucleotide sequences designed based on the SNP sites of the application of the SNP site combination of Litopenaeus vannamei for use in family mixed breeding trait evaluation of claim 1, each nucleotide sequence being 110 base pairs (bp) in length and configured to provide, on average, 55 bp of upstream sequence and 55 bp of downstream sequence flanking the SNP site.

3. A kit for use in Litopenaeus vannamei family mixed breeding trait evaluation, comprising: a probe mixture and hybridization capture reagents, wherein the probe mixture is prepared from the molecular probe of claim 2.

4. A method for identifying genotype information of Litopenaeus vannamei using an kit, the method comprising the steps of:
(a) fragmenting genomic DNA of a Litopenaeus vannamei sample, performing end-repair and adapter ligation on the fragmented DNA, purifying to obtain a sequencing library, amplifying the sequencing library, and purifying the amplified library to obtain purified library DNA;
(b) adding the probe mixture and hybridization capture reagents of the kit of claim 3 to the purified library DNA obtained in step (a), performing hybridization capture and elution to remove non-target DNA fragments and thereby enrich target DNA fragments comprising the SNP sites, and amplifying and purifying the enriched target DNA to construct a targeted sequencing library; and
(c) assessing the targeted sequencing library obtained in step (b) for qualification, sequencing the qualified targeted sequencing library using second-generation sequencing technology, and comparing the sequencing reads to a Litopenaeus vannamei reference genome sequence to obtain genotype information of the tested Litopenaeus vannamei.

5. A method for evaluating family mixed-breeding traits of Litopenaeus vannamei using a kit, the method comprising:
S1 Preparation:
1. selecting larvae from at least 10 different families;
2. taking paternal, maternal and sibling individuals of the larvae selected in step 1) as target individuals, and determining genotype information of the target individuals using the method of claim 4;
S2 Mixed breeding:
3) from each family selected in step 1), randomly selecting 30 to 50 larvae and evenly distributing the selected larvae into multiple parallel breeding pools for mixed rearing, maintaining water temperature at least 23° C., and rearing the larvae for 60 to 90 days to obtain test individuals;
S3 Trait evaluation:
4) counting the number of test individuals in each breeding pool, collecting the test individuals, and measuring body weight of the collected test individuals;
5) determining genotype information of the test individuals obtained in step 4) using the method of claim 4;
6) reconstructing pedigree relationships by combining the genotype information of the test individuals from step 5) with the genotype information of the target individuals from step 2) to assign each test individual to a family; and
7) based on the family assignments from step 6), calculating survival counts and body weight for test individuals of each family to obtain survival and growth trait data for the Litopenaeus vannamei family mixed-breeding groups.

6. The method of claim 5, wherein in step 1) the shrimp larvae are at the P15-P40 stage.

7. The method of claim 5, wherein in step 2), sibling individuals used for genotype identification are at P45 and above.

8. The method of claim 5, wherein in step 2), during the process of identifying target individuals' genotype information: if genotyping of both parents is successful, select 1-3 sibling individuals from the family for genotyping; if genotyping of one parent is unsuccessful, select 2-4 sibling individuals for genotyping; if genotyping of both parents is unsuccessful, select 3-5 sibling individuals for genotyping.

9. The method of claim 5, wherein in step 4), the stocking density of larvae from each family in each breeding pool is 100-300 individuals/m³.

10. The method of claim 5, wherein in step 6): when genotype information for both parents is available among the target individuals, pedigree reconstruction is performed using the genotype information of the test individuals together with the parents' genotype information of the target individuals, thereby assigning family membership to the test individuals; when genotype information for both parents is absent, clustering analysis is performed using the genotype information of test individuals and the sibling individuals within the target individuals, and family assignments for test individuals are inferred based on the known family identities of the sibling individuals within the target individuals.
